(19) United States
(12) Patent Application Publication
(10) Pub. No.: US 2024/0369523 A1
(43) Pub. Date: Nov. 7, 2024

(54) IN VITRO RELEASE ASSAY METHODS FOR LIPOSOMAL AMINOGLYCOSIDE FORMULATIONS

(71) Applicant: Insmed Incorporated, Bridgewater, NJ (US)

(72) Inventor: Toni SHALER, Bridgewater, NJ (US)

(21) Appl. No.: 18/568,437

(22) PCT Filed: Jun. 8, 2022
(86) PCT No.: PCT/US2022/032629

§ 371 Date: Dec. 8, 2023

Related U.S. Application Data
(60) Provisional application No. 63/208,894, filed on Jun. 9, 2021.

(51) Int. Cl. G01N 33/15; A61K 9/127; A61K 31/726

(52) U.S. Cl. CPC G01N 33/15; A61K 9/1277; A61K 31/726

(57) ABSTRACT

The present disclosure relates to in vitro release (IVR) methods for liposomal aminoglycoside formulations, in particular liposomal amikacin formulations.

### CROSS-REFERENCE TO RELATED APPLICATION

This application claims priority from U.S. Provisional Application Ser. No. 63/208,894, filed Jun. 9, 2021, incorporated by reference herein in its entirety for all purposes.

### FIELD OF THE INVENTION

The present invention relates to in vitro methods for measuring the aminoglycoside release profile from liposomal aminoglycoside formulations, in particular liposomal amikacin formulations.

### BACKGROUND OF THE INVENTION

The oral inhalation of liposomes encapsulating the antibiotic amikacin has been proposed to treat certain pulmonary infections. One such formulation, amikacin liposome inhalation suspension, or ALIS, was initially approved in 2018 by the United States Food and Drug Administration (FDA) for oral inhalation by adults who have limited or no alternative treatment options, for the treatment of Mycobacterium avium complex (MAC) lung disease as part of a combination antibacterial drug regimen in patients who do not achieve negative sputum cultures after a minimum of 6 consecutive months of a multidrug background regimen therapy.

ALIS liposomes provide localized delivery of the antibiotic at high concentrations at the site of infection, while minimizing systemic exposure and hence toxicity. The liposomes also provide a prolonged therapeutic effect and increase amikacin uptake into macrophages. Liposomal formulations containing amikacin, including certain ALIS formulations, are described in, for example, U.S. Pat. Nos. 7,544,369, 7,718,189, 8,226,975, 8,632,804, 8,642,075, 8,679,532, 8,802,137, 9,566,234, 9,827,317 and 9,895,385, each of which is incorporated herein by reference in its entirety.

Inhalation delivery of liposomes is, however, complicated by their sensitivity to shear-induced stress during nebulization, which can lead to changes in physical characteristics. Under certain conditions that affect the integrity of the liposome's lipid bilayer membrane(s) (e.g., the addition of surfactants, proteins, ethanol, osmotic stress, and/or temperature elevation), the contents inside liposomes can be induced to leak out abruptly and/or over a period of time. However, as long as the changes in characteristics are reproducible and meet acceptable criteria, they need not be prohibitive to pharmaceutical development.

According to the FDA's April 2018 Guidance for Industry, “Liposome Drug Products: Chemistry, Manufacturing and Controls; Human Pharmacokinetics and Bioavailability; and Labeling Documentation”, an in vitro release profile for the drug substance is a useful property for characterizing a liposome drug formulation, and a validated analytical procedure should be established. When a liposome drug product is extremely stable under physiological conditions, an in vitro quality control release test can be performed under non-physiological conditions to accelerate the release of drug substance from the liposomes. However, as no liposomal formulation for inhalation has been approved to date by FDA, no further guidance for developing a validated analytical test method has been provided. The present invention addresses this and other needs.

### SUMMARY OF THE INVENTION

The present invention relates to in vitro release (IVR) methods for evaluating the aminoglycoside release profile from a liposomal aminoglycoside formulation, such as a liposomal amikacin formulation, such as those suitable for inhalation.

One aspect of the IVR method for evaluating the aminoglycoside release profile from a liposomal aminoglycoside formulation is by performing a dissolution test with the liposomal aminoglycoside formulation in one or more dialysis devices which are disposed (or placed) in a dissolution medium (such as a buffer) that contains a stirring element (“stirring element” is also referred to herein as a “dissolution apparatus”). The liposomal aminoglycoside formulation comprises an aminoglycoside encapsulated in a plurality of liposomes. The one or more dialysis devices each has a predetermined volume, and each comprises a dialysis membrane having a predetermined molecular weight cut off (MWCO). The dissolution medium includes a predetermined amount of surfactant in a buffer. The dialysis membrane is permeable to the aminoglycoside, the dissolution medium, and surfactant such that two-way passage occurs across the dialysis membrane(s). Specifically, surfactant diffuses into and through the one or more dialysis membranes over time, and the surfactant increases the permeability of the liposomes to allow for free aminoglycoside release from the liposomes, and free aminoglycoside diffuses across the one or more dialysis membranes and into the dissolution medium. During the dissolution test, the stirring element is operated. In one embodiment, the stirring element is a USP dissolution apparatus 2 (paddle).

Preferably, the dialysis membrane is not permeable or is substantially impermeable to the aminoglycoside encapsulated liposomes. In a preferred embodiment, the dialysis membrane has a predetermined MWCO between about 20 kD and about 1500 kD. In a further embodiment, the dialysis membrane has a predetermined MWCO of about 1000 kD.

One embodiment of the IVR method comprises:
(a) operating a dissolution apparatus in a dissolution vessel, the dissolution vessel containing (i) a predetermined quantity of the liposomal aminoglycoside formulation in one or more dialysis devices, each having a predetermined volume and a dialysis membrane having a predetermined MWCO, and (ii) a dissolution medium having a predetermined quantity of surfactant, wherein each dialysis membrane is permeable to the aminoglycoside, the dissolution medium, and the surfactant, and wherein the one or more dialysis devices are immersed in the dissolution medium;
(b) after a predetermined time interval, removing an aliquot of the dissolution medium;
(c) analyzing the aliquot for free aminoglycoside content; and
(d) optionally, repeating steps (b) and (c) after one or more additional time intervals.

The aliquot removed from the dissolution medium can be subjected to solid phase extraction (such as a cation exchange sorbent material) to remove surfactant, prior to analysis of the aliquot for free aminoglycoside content.

In one embodiment, step (b) is performed a total of three, four, five, or six times. In yet another embodiment, step (b) is performed four times. In a further embodiment, the liposomal aminoglycoside formulation is an ALIS formulation. The liposomal aminoglycoside formulation, in one embodiment, comprises an aminoglycoside or a pharmaceutically acceptable salt thereof encapsulated in a plurality of liposomes. The lipid component of the liposomes in one embodiment comprises electrically neutral lipids. In a further embodiment, the electrically neutral lipids consist of dipalmitoylphosphatidylcholine (DPPC) and cholesterol.

In one embodiment, analyzing the aliquot for free aminoglycoside content comprises performing high performance liquid chromatography (HPLC) on the aliquot. HPLC in a further embodiment, is carried out with evaporative light scattering detection (ELSD) or charged aerosol detection (CAD) to determine the free aminoglycoside content. In a further embodiment, HPLC is employed to determine total aminoglycoside content of the aliquot.

In one embodiment, step (a) of operating a dissolution apparatus is carried for a time sufficient to (i) allow the surfactant to diffuse into and through the one or more dialysis membranes and (ii) to allow free aminoglycoside to diffuse through the one or more dialysis membranes and into the dissolution medium. The dissolution apparatus, in a further embodiment, is the USP apparatus 2 (paddle).

In another embodiment, step (a) of operating a dissolution apparatus comprises disposing or placing the one or more dialysis devices in the dissolution medium within the dissolution vessel containing the dissolution apparatus (e.g., USP Apparatus 2 (Paddle) or a stir bar); and is carried out for a time sufficient to (i) allow the surfactant to diffuse into and through the one or more dialysis membranes and (ii) to allow free aminoglycoside to diffuse through the one or more dialysis membranes and into the dissolution medium.

In one embodiment, the one or more dialysis devices are immersed in the dissolution medium and are buoyant in the dissolution medium. The one or more dialysis devices can be fully immersed or partially immersed in the dissolution medium and do not interfere with the dissolution apparatus when it is operated.

In another aspect of the IVR method for evaluating the aminoglycoside release profile from a liposomal aminoglycoside formulation, the method includes adding predetermined quantities of a surfactant (for example, as a mixture of a surfactant and buffer (e.g., a dissolution medium comprising a surfactant and buffer)) at predetermined times to the liposomal aminoglycoside formulation to form release solutions at each predetermined time, and at a predetermined interval of time (e.g., 30 minutes) after each addition, removing an aliquot of the release solution and analyzing each aliquot for total and free aminoglycoside content. The method may further include at one or more predetermined times after the final addition of surfactant, removing and analyzing an aliquot of the release solution for total and free aminoglycoside content. Analyzing for total and free aminoglycoside content, in one embodiment, is carried out via HPLC.

In one embodiment of this aspect, the IVR method comprises:
(a) adding a predetermined quantity of dissolution medium comprising a surfactant and buffer to a predetermined quantity of the liposomal aminoglycoside formulation to obtain a release solution;
(b) after a predetermined time interval, removing an aliquot of the release solution and analyzing the aliquot for total and free aminoglycoside content;
(c) increasing the ratio of surfactant to the liposomal aminoglycoside formulation in the release solution (for example, by addition of a dissolution medium);
(d) after a predetermined time interval, removing an aliquot of the release solution and analyzing the aliquot for total and free aminoglycoside content;
(e) optionally, repeating steps (c) and (d) one or more times; and
(f) optionally repeating step (d) one or more times.

In one embodiment, step (e) is performed a total of three, four, five, or six times. In yet another embodiment, step (e) is performed three times and then step (f) is performed once. In a further embodiment, the liposomal aminoglycoside formulation is an ALIS formulation.

The IVR methods described herein are useful for the characterization of liposomal aminoglycoside formulations and manufacturing conditions used to make the liposomal aminoglycoside formulations, including liposomal amikacin formulations. The aminoglycoside can be in free base form or salt form, e.g., an aminoglycoside sulfate salt (e.g., amikacin sulfate). The release profile produced by the IVR methods described herein is reflective of a product that actively leaks over a period of time (not instantaneously) and demonstrates the ability to completely release the encapsulated aminoglycoside. The IVR methods described herein can be completed within a normal workday, are discriminatory (i.e., they are capable of distinguishing between batches of liposomal aminoglycoside formulations that have significantly different release rates from those with satisfactory quality, for example, because of differences in lipid-to-aminoglycoside weight ratio), and are adaptable for implementation in a quality control environment. Furthermore, the conditions and media used in the methods have physiological relevance.

A liposomal aminoglycoside formulation's typical leakage profile under specified conditions can be established with the IVR method, and then used to verify the consistency in the quality of liposomal aminoglycoside formulations manufactured in different batches, by different processes, or at different scales. Preferably, the leakage at the beginning of the test is low whereas the leakage at the end of the test approaches about 100%, or complete leakage.

### DETAILED DESCRIPTION OF THE INVENTION

Aspects of the present invention are directed to in vitro release (IVR) methods for assessing aminoglycoside release from liposomal aminoglycoside formulations in which the aminoglycoside is encapsulated within a plurality of liposomes. In embodiments described herein, the IVR methods have one or more of the following characteristics:
The IVR method is not representative of an in vivo release profile of an aminoglycoside from a liposomal aminoglycoside formulation.
The IVR profile obtained from the method is reflective of a liposomal product that actively releases aminoglycoside over time.
The IVR method demonstrates the ability to release all or substantially all of the aminoglycoside from the liposomal aminoglycoside formulation.
The conditions employed in the IVR method have some physiological reference.
The IVR method is adaptable for implementation in a quality control environment.
The IVR method can be completed within a normal workday.
The IVR method does not induce an initial burst of aminoglycoside release from the liposomal formulation, as this could mask evidence of “dose dumping” of the formulation.

The IVR methods described herein are based in part on an increase in permeability of liposomes to aminoglycoside, e.g., amikacin, due to surfactant binding and/or surfactant interaction with the liposomal membrane. Unlike other methods, the aminoglycoside release due to the IVR methods described herein is not based on liposomal membrane rupture. It has been found that the use of serum to lyse the liposomal membrane is insufficient to induce near complete aminoglycoside release from net neutral charged liposomes.

Surprisingly, elevated temperature and lower osmotic pressure, which have served as a basis for prior IVR methods, were inadequate to sufficiently increase the permeability of the lipid membranes of the liposomes to affect complete aminoglycoside release from the liposomal aminoglycoside formulations set forth herein. Consequently, in the methods provided herein, an active liposomal membrane disruption to increase permeability is employed. The active disruption techniques employed herein increase membrane permeability without membrane rupture. In one embodiment, the active disruption occurs through the addition of a surfactant to the liposomal aminoglycoside formulation.

Moreover, there is biological relevance to using a surfactant disruptive agent to accelerate the release of aminoglycoside from liposomal aminoglycoside formulations. Although the exact molecular interactions between liposomes and biological factors have not been elucidated fully, it was found that the release of aminoglycoside from liposomal aminoglycoside formulations was accelerated significantly upon incubation ex vivo with a liquefied Pseudomonas-infected sputum sample from a cystic fibrosis patient (Meers et al. (2008). Journal of Antimicrobial Chemotherapy 61, pp. 859-868, the disclosure of which is incorporated by reference herein in its entirety for all purposes). A substantial portion of this activity was found to be associated with two small organic-solvent soluble molecules: (i) mono-rhamnolipid and (ii) di-rhamnolipid.

In one aspect of the invention, an IVR method is provided. In one embodiment of this aspect, the liposomal aminoglycoside formulation is present in one or more dialysis devices, each having a predetermined volume and a dialysis membrane. The predetermined volume of one dialysis device, in one embodiment, is 5 mL. In another embodiment, the predetermined volume of one dialysis device, is 10 mL. The one or more dialysis devices are present in a vessel having a dissolution apparatus and a dissolution medium disposed therein. The dissolution medium includes a surfactant. In embodiments described herein, the one or more dialysis devices are immersed or substantially immersed in the dissolution medium, while also buoyant in the dissolution medium. The dissolution apparatus is operated, and the surfactant diffuses through the dialysis membrane(s) and induces release of the aminoglycoside from the liposomes. The free aminoglycoside then diffuses through the dialysis membrane into the external dissolution medium from which aliquots are taken at predetermined time intervals, and tested for free aminoglycoside content. Selection of an appropriate molecular weight cut off (MWCO) dialysis membrane prevents diffusion of the liposomes into the external dissolution medium while allowing for the sampling of only free amikacin from the external dissolution medium.

In one embodiment, analyzing the aliquot for free aminoglycoside content comprises performing high performance liquid chromatography (HPLC) on the aliquot. HPLC in a further embodiment, is carried out with evaporative light scattering detection (ELSD) or charged aerosol detection (CAD) to determine the free aminoglycoside content. In a further embodiment, HPLC is carried out to determine total aminoglycoside content.

In another aspect, an IVR method is provided that is based in part on a stepwise approach where additional surfactant is added over time to the liposomal aminoglycoside formulation. Without wishing to be bound by theory, this approach is thought to simulate the continuous buildup of proteins and other biological factors interacting with the liposome membranes once the liposomal aminoglycoside formulation is administered to a patient; and to facilitate complete aminoglycoside release within a timeframe appropriate for quality control analysis. In one embodiment of this aspect, the timeframe appropriate for quality control analysis is from about 2 hours to about 5 hours, or from about 2.5 hours to about 5 hours, or from about 3 hours to about 5 hours, or from about 2.5 hours to about 4 hours. In a further embodiment, the timeframe appropriate for quality control analysis is about 3 hours. The timeframe appropriate for quality control analysis, in one embodiment, is a timepoint that allows for the subsequent immediate processing and testing of the resulting aliquots of release solution to occur within the same day in order to avoid storage and potential changes in the composition of the post release aliquots.

Additional aspects and embodiments of the invention are discussed in detail below.

### Definitions

Unless defined otherwise, all technical and scientific terms used herein generally have the same meaning as commonly understood by one of ordinary skill in the art to which this invention belongs.

The term “comprising” is open ended and, in connection with a composition, refers to the elements recited. The term “comprising” as used in connection with the compositions described herein can alternatively cover compositions “consisting essentially of” or “consisting of” the recited components.

As used herein, the term “liposome(s)” refers to completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “head” orient towards the aqueous phase.

The term “aminoglycoside encapsulated liposome” or “liposomal aminoglycoside” refers to a liposome encapsulating an aminoglycoside, where the aminoglycoside can be in any form, such as a salt form or free base form. The aminoglycoside in one embodiment, is amikacin or amikacin sulfate. In embodiments described herein, a liposomal aminoglycoside formulation comprises an aminoglycoside encapsulated in a plurality of liposomes.

The term “amikacin encapsulated liposome” or “liposomal amikacin” refers to a liposome encapsulating amikacin, where the amikacin may be in any form (such as salt forms). In one embodiment, the liposome encapsulates amikacin sulfate. In embodiments described herein, a liposomal amikacin formulation comprises an amikacin encapsulated in a plurality of liposomes.

The term “encapsulated” refers to the aminoglycoside being associated with the liposome, either (i) as part of a complex with the liposome, (ii) within the aqueous phase of the liposome, (iii) within the hydrophobic liposomal bilayer, (iv) at the interfacial head group region of the liposomal bilayer, or (v) a combination thereof.

The term “amikacin liposome inhalation suspension” or “ALIS” refers to an amikacin encapsulated liposome formulation where the amikacin is present as amikacin sulfate and the lipid component of the liposomes consists of dipalmitoylphosphatidylcholine (DPPC) and cholesterol. ALIS includes amikacin at a concentration of about 70 mg/mL (expressed as amikacin base, e.g., 70 mg/mL+10%), about 40 to 56 mg/mL total lipid (DPPC and cholesterol) (e.g., about 47 mg/mL total lipid, and DPPC and cholesterol in an about 2:1 weight ratio (DPPC: cholesterol), and a lipid-to-amikacin weight ratio of about 0.60 (lipid): 1 (amikacin) to about 0.80 (lipid): 1 (amikacin) (such as from about 0.65 (lipid): 1 (amikacin) to about 0.75 (lipid): 1 (amikacin).

The term “dissolution medium” as used herein comprises a surfactant and a buffer and in some instances is referred to herein as a “surfactant/buffer mixture”. In one embodiment, a surfactant is mixed with a buffer to obtain the dissolution medium. In one embodiment, the dissolution medium is equilibrated prior to use.

Described herein are methods for evaluating the aminoglycoside release profile from a liposomal aminoglycoside formulation. The liposomal aminoglycoside formulation subjected to the methods described herein comprise an aminoglycoside, or a pharmaceutically acceptable salt thereof, encapsulated in a plurality of liposomes. In one embodiment, the lipid component of the plurality of liposomes consists of electrically neutral lipids.

In one aspect of the IVR methods described herein, one or more dialysis devices, a dissolution apparatus, and a dissolution medium are employed. The liposomal aminoglycoside formulation is present in the one or more dialysis devices, and each dialysis device has a predetermined volume and a dialysis membrane. The liposomal aminoglycoside formulation is present in the one or more dialysis devices and the device(s) are placed into a vessel containing (i) a surfactant enriched dissolution medium and, (ii) a dissolution apparatus immersed in the dissolution medium. The one or more dialysis devices, in one embodiment, are immersed and buoyant in the dissolution medium and do not interfere with the dissolution apparatus when the dissolution apparatus is operated. Without wishing to be bound by theory, two-way passage occurs across the one or more dialysis membranes during the operation of the dissolution apparatus, e.g., surfactant diffuses into the one or more dialysis devices over time through the dialysis membrane(s), which renders the liposomal bilayers of the liposomes permeable and induces free aminoglycoside release. The free aminoglycoside diffuses through the dialysis membrane(s) and into the dissolution medium over time. The dissolution apparatus is operated, and aliquots of the dissolution medium are taken at time intervals for analysis of free aminoglycoside levels. The method may also employ a solid phase extraction (SPE) process to remove excess surfactant from the aliquots prior to free aminoglycoside analysis.

In one embodiment, the dialysis device (e.g., one dialysis device or two dialysis devices) are inserted through the center of a flotation ring(s) to maintain buoyancy in the dissolution medium.

In one embodiment of this aspect, the IVR method is performed with agitation with a dissolution apparatus, for example with a United States Pharmacopeia (USP) Apparatus 2 (paddle) (see United States Pharmacopeia (USP) General Chapters Dissolution (2011), the disclosure of which is incorporated by reference in its entirety for all purposes), or with a stir plate with stir bar. In a further embodiment, the IVR method is conducted at a controlled temperature, e.g., from about 30° C. to about 40° C., from about 32° C. to about 40° C., from about 34° C. to about 40° C. or from about 36° C. to about 40° C. In a preferred embodiment, the IVR method is performed at 37° C.±3° C. In one embodiment, the dissolution apparatus is operated at from about 75 to about 150 rpm (e.g., at 150 rpm). In one embodiment, the dissolution apparatus comprises a paddle or a stir bar and is operated at from about 75 rpm to about 150 rpm (e.g., at 150 rpm).

The surfactant, in one embodiment, is added at a concentration that is above its critical micelle concentration (CMC).

The predetermined volume of a dialysis device, in one embodiment, is 5 mL. In another embodiment, the predetermined volume of a dialysis device, is 10 mL. In yet another embodiment, the predetermined volume of a dialysis device, is from about 4 mL to about 11 mL, or from about 5 mL to about 10 mL.

In one embodiment, the IVR method comprises:
(a) operating a dissolution apparatus in a dissolution vessel, the dissolution vessel containing (i) a predetermined quantity of the liposomal aminoglycoside formulation in one or more dialysis devices and (ii) a dissolution medium having a predetermined quantity of surfactant (e.g., 5% Triton X-100 in DPBS), wherein each dialysis membrane is permeable to the aminoglycoside, the dissolution medium, and the surfactant and the dissolution apparatus is immersed in the dissolution medium in the dissolution vessel;
(b) after a predetermined time interval, removing an aliquot of the dissolution medium;
(c) analyzing the aliquot for free aminoglycoside content; and
(d) optionally, repeating steps (b) and (c) after one or more additional time intervals.

In one embodiment, operating a dissolution apparatus comprises agitating the dissolution medium. In a further embodiment, the agitating is carried out with a dissolution apparatus comprising a paddle (e.g., the USP apparatus 2). In another embodiment, the agitating is carried out with a dissolution apparatus comprising a stir bar. In a further embodiment, the dissolution vessel is placed on a stir plate and prior to operating the stir bar.

In one embodiment, step (a) of operating a dissolution apparatus is carried for a time sufficient to (i) allow the surfactant to diffuse into and through the one or more dialysis membranes and (ii) to allow free aminoglycoside to diffuse through the one or more dialysis membranes and into the dissolution medium.

In another embodiment, step (a) of operating a dissolution apparatus comprises disposing or placing the one or more dialysis devices in the dissolution medium within the dissolution vessel containing the dissolution apparatus (e.g., USP Apparatus 2 (paddle) or stir bar); and the dissolution apparatus is operated for a time sufficient to (i) allow the surfactant to diffuse into and through the one or more dialysis membranes and (ii) to allow free aminoglycoside to diffuse through the one or more dialysis membranes and into the dissolution medium. Where a stir bar is employed as the dissolution apparatus, it should be understood that the vessel is placed on a stir plate to operate the dissolution apparatus.

Prior to operating the dissolution apparatus, the following steps may be performed:
(i) adding a predetermined quantity of the liposomal aminoglycoside formulation into one or more dialysis devices; and
(ii) placing the one or more dialysis membranes into the dissolution vessel having the dissolution medium comprising a predetermined quantity of the surfactant.

In one embodiment, the surfactant is equilibrated in a dissolution vessel prior to step (a). Equilibration can occur with or without agitation. In a further embodiment, the initial equilibration is carried out at about 30° C. to about 40° C., e.g., from about 35° C. to about 40° C., or about 37° C.

In one embodiment, the liposomal aminoglycoside formulation is equilibrated to ambient temperature, e.g., for at least about 45 minutes prior to adding the formulation to the one or more dialysis devices or operating the dissolution apparatus. In a further embodiment, prior to step (a), the liposomal aminoglycoside formulation is shaken and/or vortexed until it appears by visual inspection to be uniform and well mixed. In a further embodiment, the liposomal aminoglycoside formulation is transferred into a separate dissolution vessel to provide the predetermined quantity of liposomal aminoglycoside formulation used in step (a), and is equilibrated with agitation (e.g., USP dissolution apparatus with paddle or a stir plate with stir bar). Equilibration, in one embodiment, is carried out for about 30, about 60, about 90, about 120, or about 150 minutes. In even a further embodiment, equilibration with agitation is carried out at about 30° C. to about 40° C., e.g., from about 35° C. to about 40° C., or about 37° C.

In one embodiment, prior to step (a), the liposomal aminoglycoside formulation and/or the dissolution medium (comprising surfactant in buffer) are equilibrated in separate dissolution vessels, e.g., for at least 30 min. In one embodiment, the liposomal aminoglycoside formulation is equilibrated to ambient temperature (e.g., for at least 45 min). In one embodiment, the dissolution medium is equilibrated to about 37° C. with from about 75 rpm to about 150 rpm agitation with a paddle or stir bar (e.g., for at least 30 min). In a further embodiment, an evaporation cover is placed on the vessel comprising the dissolution medium during the equilibration.

In one embodiment, the dialysis membrane is composed of a cellulose ester. In one embodiment, the one or more dialysis devices are one or more Float-A-Lyzer dialysis devices (e.g., available from Repligen Corp., Rancho Dominguez, California, USA). In one embodiment, at least about 80%, at least about 85%, at least about 90%, at least about 95% or at least about 99%, of the free aminoglycoside (such as amikacin) diffuses across the membrane within about 12 or 24 hours during the IVR method.

In one embodiment, the dialysis membrane has a molecular weight cut off (MWCO) of between about 20 kD and about 1500 kD, such as between about 100 kD and about 1000 kD, e.g., about 20 kD, about 100 kD, about 300 kD or about 1000 kD. In a preferred embodiment, the dialysis membrane has a MWCO of about 1000 kD. Preferably, the dialysis membrane has a MWCO that permits the free aminoglycoside, surfactant, and dissolution medium to freely move across the dialysis membrane while preventing the liposomes, or substantially preventing the liposomes, from crossing the membrane.

In one embodiment, one dialysis device is used in the IVR method. In a further embodiment, the dialysis device comprises a cellulose ester membrane having a MWCO of about 1000 kD. The cellulose ester membrane can comprise a single ester species or a plurality of ester species.

In one embodiment, the IVR method comprises (a) adding a predetermined quantity of the liposomal aminoglycoside formulation into two dialysis devices, such as two Float-A-Lyzer dialysis devices (e.g., each having a MWCO of about 1000 kD). In a further embodiment, each dialysis device has a predetermined volume of about 5 mL or about 10 mL. In another embodiment, each dialysis device has a predetermined volume of from about 5 mL to about 10 mL.

In an embodiment where two dialysis devices are employed, each device has a predetermined volume of about 5 mL and a cellulose ester membrane. In a further embodiment, the volume of the liposomal aminoglycoside formulation is about 8 mL to about 10 mL and is split about evenly into each device. In a further embodiment, the volume of the liposomal aminoglycoside formulation is from about 8 mL to about 9 mL, e.g., about 8.4 mL. Where two dialysis devices are employed, the liposomal aminoglycoside formulation subjected to the IVR method may be split evenly or substantially evenly between the two devices. In one embodiment, a single, bundled flotation ring is used to keep both dissolution devices buoyant in the dissolution medium while the dissolution apparatus is being operated.

Aliquots can be removed at various time points, such as 1, 2, 6, and 24 hours after initiating the dissolution test. In another embodiment, aliquots are removed at 1, 2, 3, 6, and 24 hours after initiating the dissolution test. In even another embodiment, aliquots are removed at 1, 2, 4, 6, and 24 hours after initiating the dissolution test. In yet another embodiment, aliquots are removed at 1, 2, 3, 4, 6, and 24 hours after initiating the dissolution test. In even another embodiment, aliquots are removed at 1, 3, 4, 6, and 24 hours after initiating the dissolution test. In yet even another embodiment, aliquots are removed at 1, 2, 3, 4, and 24 hours after initiating the dissolution test. In one embodiment, an aliquot removed from the dissolution medium in step (b) is subjected to solid phase extraction (SPE) prior to analyzing the aliquot for free aminoglycoside content. The SPE may be used to remove excess surfactant prior to analysis of an aliquot. The SPE can be performed with a cation exchange sorbent material.

In one embodiment, the SPE filter (e.g., an SPE cartridge) comprises a strong cationic exchange material, such as, e.g., Oasis MCX (1 cc/30 mg) (available from Waters of Milford, MA). Without wishing to be bound by theory, the inventors theorize that protonated aminoglycoside (such as amikacin) is retained on the SPE column, while surfactant (e.g., Triton X-100, such as 5% Triton X-100 in PBS) may be washed away. The aminoglycoside (such as amikacin) may then be eluted with a basic eluting solution to afford an aminoglycoside (such as amikacin) aliquot that may subsequently be analyzed. The general SPE process is shown in the diagram below.

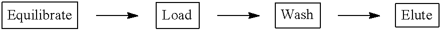

After the SPE, the amount of free aminoglycoside (e.g., amikacin) can be determined by HPLC analysis.

Some of the preferred parameters for this IVR method are as follows: (i) dissolution apparatus: USP apparatus 2 (paddle) operated at 150 RPM, (ii) dissolution medium: 5% octylphenol ethoxylate (Triton X-100) in DPBS 1× is maintained at about 37° C., (iii) the medium volume is about 900 mL with a pH of about 7.1 in the dissolution vessel, (iv) two Float-A Lyzer dialysis devices with 1000 kD MWCO membrane (cellulose ester membrane) are used per vessel to enable testing of the complete contents of one vial of ALIS drug product (i.e., entire dose of 8.4 mL), and (v) SPE is performed on sample pulls (aliquots) prior to free aminolgycoside analysis by HPLC to remove octylphenol ethoxylate (Triton X-100) from the aliquots.

In another preferred embodiment, the IVR method comprises:
(a) adding a predetermined quantity of the liposomal aminoglycoside (e.g., amikacin) formulation into a dialysis device comprising a cellulose ester membrane;
(b) placing the dialysis device into a dissolution vessel containing a dissolution medium comprising a predetermined quantity of surfactant (e.g., octylphenol ethoxylate) in a buffer (e.g., phosphate-buffered saline or Dulbecco's phosphate-buffered saline);
(c) allowing the surfactant to diffuse through the dialysis membrane and into the liposomal aminoglycoside (e.g., amikacin) formulation, thereby facilitating release of the aminoglycoside from the liposomes;
(d) allowing the free amikacin to diffuse through the cellulose ester membrane and into the dissolution medium;
(e) removing one or more aliquots of the dissolution medium at predetermined time(s) (such as at four (4) or more of the following time points: 1, 2, 3, 4, 6, 12 and 24-hours); and
(f) analyzing the aliquots for free aminoglycoside (e.g., amikacin) content.

Prior to analysis for free aminoglycoside content, the aliquots can be subjected to SPE to remove the surfactant, such as with a cation exchange sorbent material. The vessel, in one embodiment, comprises a dissolution apparatus which is operated once the dialysis device is placed in the dissolution vessel.

One preferred embodiment is a method for measuring the release profile of aminoglycoside from a liposomal aminoglycoside formulation comprising aminoglycoside encapsulated in a plurality of liposomes, the method comprising:
(a) adding a predetermined quantity of the liposomal aminoglycoside formulation in one dialysis device or divided into two or more dialysis devices, wherein each dialysis device has a predetermined volume and a dialysis membrane comprised of a cellulose ester or a combination of cellulose esters;
(b) placing the dialysis device or devices into a dissolution vessel comprising a predetermined quantity of octylphenol ethoxylate in Dulbecco's phosphate-buffered saline and a dissolution apparatus;
(c) operating the dissolution apparatus within the dissolution vessel, wherein the dissolution apparatus is a USP apparatus 2 (paddle) operated at 37° C. at about 150 rpm;
(d) after a predetermined time interval, removing an aliquot of the dissolution medium;
(e) analyzing the aliquot for free aminoglycoside content; and
(f) optionally, repeating steps (d) and (e) after one or more additional time intervals.

In another aspect of the invention, the IVR method employs a stepwise approach where surfactant is initially added to a predetermined quantity of the liposomal aminoglycoside formulation to obtain a release solution; and additional surfactant is added to the release solution at one or more subsequent timepoints in order to increase the surfactant to lipid ratio.

In one embodiment of this aspect, the IVR method comprises:
(a) adding a predetermined quantity of dissolution medium comprising a surfactant and buffer to a predetermined quantity of the liposomal aminoglycoside formulation to obtain a release solution;
(b) after a predetermined time interval, removing an aliquot of the release solution and analyzing the aliquot for total and free aminoglycoside content;
(c) increasing the ratio of surfactant to the liposomal aminoglycoside formulation in the release solution (e.g, by surfactant addition, such as by the addition of a dissolution medium);
(d) after a predetermined time interval, removing an aliquot of the release solution and analyzing the aliquot for total and free aminoglycoside content;
(e) optionally, repeating steps (c) and (d) one or more times; and
(f) optionally, repeating step (d) one or more times.

Each addition of the surfactant can be performed, for instance, by adding a dissolution medium comprising a surfactant in a buffer. In one embodiment, each addition of surfactant is with the same dissolution medium, although the amount (volume) of the dissolution medium may vary for each addition. In another embodiment, each addition of surfactant is with a mixture containing the same surfactant and same buffer, although (i) the amount of the dissolution medium and/or (ii) the amount of surfactant in dissolution medium may vary for each addition.

In one embodiment, the method is carried out while operating a dissolution apparatus within the vessel.

The predetermined quantity of liposomal aminoglycoside formulation, in one embodiment, is a predetermined quantity that is pooled from two or more vials of the formulation originating from the same manufacturing batch. Without wishing to be bound by theory, it is thought that because the liposomal aminoglycoside formulation is a homogeneous suspension, a composite of multiple vials is comparably reflective of the respective batch.

In a further embodiment, step (e) is performed one time, two times, three times, or four times. In a further embodiment, step (e) is performed three times.

In one embodiment, the liposomal aminoglycoside formulation is an ALIS formulation.

Stepwise increase of the ratio of surfactant to liposomal aminoglycoside formulation in the release solution is designed in one embodiment to cause a generally logarithmic curve of the ratio of surfactant to lipid with successive additions. The volume of added surfactant therefore, in one embodiment, is increased each time step (c) is carried out. The increase in surfactant volume in one embodiment, is added to provide the surfactant concentration sufficient and/or necessary to increase liposome membrane permeability.

In another embodiment, the IVR method comprises:
(a) adding a predetermined quantity of dissolution medium comprising a surfactant in a buffer to a predetermined quantity of the liposomal aminoglycoside formulation to obtain a release solution;
(b) after a predetermined time interval, removing an aliquot of the release solution and analyzing the aliquot for total and free aminoglycoside content;
(c) adding a predetermined quantity of the dissolution medium to the release solution;
(d) after a predetermined time interval, removing an aliquot of the release solution and analyzing the aliquot for total and free aminoglycoside content;
(e) optionally repeating steps (c) and (d) one or more times; and
(f) optionally, repeating step (d) one or more times.

In another embodiment, the IVR method comprises:
(a) adding 0.5 mL of a dissolution medium into 30 mL of the liposomal aminoglycoside formulation in a vessel comprising a dissolution apparatus to form a release solution;
(b) 30 minutes after step (a) [time=0.5 hour], (i) removing and analyzing 3 mL of the release solution for total and free aminoglycoside content, and (ii) adding 1.7 mL of dissolution medium to the remainder of the release solution;
(c) 30 minutes after step (b) [time=1 hour], (i) removing and analyzing 3 mL of the release solution for total and free aminoglycoside content, and (ii) adding 4.0 mL of dissolution medium to the remainder of the release solution;
(d) 30 minutes after step (c) [time=1.5 hours], (i) removing and analyzing 3 mL of the release solution for total and free aminoglycoside content, and (ii) adding 16 mL of dissolution medium to the remainder of the release solution;
(e) 30 minutes after step (d) [time=2 hours], (i) removing and analyzing 20 mL of the release solution for total and free aminoglycoside content, and (ii) adding 100 mL of dissolution medium to the remainder of the release solution;
(f) 30 minutes after step (e) [time=2.5 hours], removing and analyzing 20 mL of the release solution for total and free aminoglycoside content; and
(g) 30 minutes after step (f) [time=3 hours], removing and analyzing 20 mL of the release solution for total and free aminoglycoside content,
wherein the dissolution medium comprises octylphenol ethoxylate (e.g., as Triton™ X-100) in phosphate-buffered saline (PBS) (such as Dulbecco's phosphate-buffered saline) and the dissolution apparatus is operated during the method. In a further embodiment, the aminoglycoside is amikacin or a pharmaceutically acceptable salt thereof, e.g., amikacin sulfate. In even a further embodiment, the dissolution apparatus is a USP apparatus 2 (paddle), and the method is performed at about 37° C. (e.g., 37° C.±3° C.) at 150 (±20) rpm. In one embodiment, the octylphenol ethoxylate in PBS is 200 ppm octylphoneol ethoxylate in PBS pH 7.2 or 7.4. In a further embodiment, the PBS pH is about 7.2.

In one embodiment, the dissolution medium is equilibrated in a dissolution vessel prior to step (a), with or without agitation. In a further embodiment, the initial equilibration is at about 30° C. to about 40° C., e.g., from about 35° C. to about 40° C., or about 37° C.

In one embodiment of the IVR method provided herein, the liposomal aminoglycoside formulation is equilibrated to ambient temperature, e.g., for at least about 45 min. prior to step (a). In a further embodiment, prior to step (a), the liposomal aminoglycoside formulation is shaken and/or vortexed until it appears uniform and well mixed. In a further embodiment, the liposomal aminoglycoside formulation is transferred into a separate dissolution vessel to provide the predetermined quantity of liposomal aminoglycoside formulation used in step (a), and is equilibrated with agitation with a stirring element (e.g., USP dissolution apparatus with paddle or a stir plate with stir bar). Equilibration, in one embodiment, is carried out for about 30 min., about 60 min., about 90 min., about 120 min., or about 150 min. In even a further embodiment, equilibration with agitation is carried out at about 30° C. to about 40° C., e.g., from about 35° C. to about 40° C., or about 37° C.

In a further embodiment of one of the methods described herein, prior to step (a), the liposomal aminoglycoside formulation and/or the dissolution medium (comprising surfactant in buffer) are equilibrated in separate dissolution vessels, e.g., for at least 30 min. In one embodiment, the liposomal aminoglycoside formulation is equilibrated to ambient temperature (e.g., for at least 45 min). In one embodiment, the dissolution medium is equilibrated to about 37° C. with from about 75 RPM to about 150 RPM agitation with a stirring element such as a paddle or stir bar (e.g., for at least 30 min). In a further embodiment, an evaporation cover is placed on the vessel during equilibration.

In the methods described herein, the surfactant, present in the dissolution medium, can be any type of surfactant, such as an anionic surfactant or non-ionic surfactant. The surfactant, in one embodiment, is non-ionic. In a further embodiment, the surfactant comprises a hydrophilic polyethylene oxide chain. In even a further embodiment, the surfactant comprising a hydrophilic polyethylene oxide chain further comprises an aromatic hydrocarbon group that is lipophilic or hydrophobic. In one embodiment, the surfactant comprises octylphenoxypolyethoxyethanol (sold under the trade names Nonidet P-40 or IGEPAL CA). In one embodiment, the surfactant is a non-ionic surfactant. In a further embodiment, the non-ionic surfactant is octylphenol ethoxylate (available as Triton™ X-100 from Sigma Aldrich of St. Louis, MO). Suitable anionic surfactants for use in the dissolution media employed herein include rhamnolipids. In one embodiment, the surfactant has a critical micelle concentration (CMC) less than about 5 mM, less than about 2 mM, or less than about 1 mM. For instance, the nonionic surfactant may have a CMC of about 0.1 mM, 0.2 mM, 0.3 mM, 0.4 mM, or 0.5 mM, or a CMC ranging from about 0.1 to about 1 mM, such as from about 0.1 to about 0.5 mM.

The dissolution medium contains the surfactant and a buffer. In one embodiment, the surfactant is dissolved in a buffer to provide a dissolution medium. Suitable buffers include, but are not limited to, phosphate-buffered saline such as Dulbecco's phosphate-buffered saline (DPBS). In one preferred embodiment, the dissolution medium is octylphenol ethoxylate in DPBS. The dissolution medium may be free, or substantially free (e.g., contains less than about 5%, such as less than about 4%, less than about 3%, less than about 2%, less than about 1%, less than about 0.5%, less than about 0.1%, less than about 0.05%, or less than about 0.01% v/v) of an alcohol, such as, e.g., ethanol.

In one embodiment, the dissolution medium used in the methods involving use of a dialysis membrane comprises from about 0.5 to about 10% v/v, from about 1% to about 5% or 6% (v/v) Triton X-100 (octylphenol ethoxylate) in phosphate-buffered saline (PBS) (such as Dulbecco's phosphate-buffered saline, DPBS). In one embodiment, the dissolution medium comprises about 5% (v/v) octylphenol ethoxylate in phosphate-buffered saline (PBS) (such as Dulbecco's phosphate-buffered saline). In a preferred embodiment, the dissolution medium is 5% (v/v) octylphenol ethoxylate in phosphate-buffered saline (PBS) (such as Dulbecco's phosphate-buffered saline) that does not contain magnesium and calcium (e.g., phosphate-buffered saline (PBS) (such as Dulbecco's phosphate-buffered saline DPBS 1×).

The methods are preferably performed with a stirring element (e.g., paddle, stir bar) in the release solution or dissolution medium. In a preferred embodiment, the method is performed with a USP apparatus 2 (paddle) at about 37° C. operated at about 150 rpm (i.e., the release solution/dissolution medium is in a vessel comprising the USP apparatus 2 (paddle) being operated at 37° C. at 150 rpm±20 rpm). In one embodiment, the IVR method is carried out in a 150 mL vessel. In one embodiment, the dissolution medium has a pH of from about 7.1 or 7.2 to about 7.4. In one embodiment, the dissolution medium has a pH of about 7.2. In another embodiment, the dissolution medium has a pH of about 7.4.

In a preferred embodiment of any of the methods described herein, the aminoglycoside is amikacin or a pharmaceutically acceptable salt thereof, such as amikacin sulfate.

In a preferred embodiment of any of the methods described herein, the dissolution medium comprises octylphenol ethoxylate in PBS, such as DPBS, for instance at a pH of about 7.1, about 7.2 or about 7.4. In a further embodiment of any of the methods described herein, the dissolution medium comprises from about 20 to about 600 ppm of octylphenol ethoxylate in DPBS, such as about 100, 150, 200, 250, 300, 350, or 400 ppm of octylphenol ethoxylate. The octylphenol ethoxylate may have an ethoxylate moles content of from about 4 to about 16, such as about 8 to about 12. In one preferred embodiment, the octylphenol ethoxylate has an ethoxylate moles content of about 9.5. In another preferred embodiment, the dissolution medium comprises about 200 ppm of octylphenol ethoxylate having an ethoxylate moles content of about 9.5 in DPBS. The dissolution medium, in one embodiment, comprises 5% surfactant in buffer (by volume), for example, 5% octylphenol ethoxylate in PBS.

In a further embodiment of any of the methods described herein, the aliquots are stored at about 2-8° C. prior to further analysis, e.g., prior to analysis for free and/or total aminoglycoside (e.g., amikacin) content.

In a further embodiment of any of the methods described herein, the removed aliquots of release solution are tested by HPLC analysis to determine free and optionally total amikacin content within 1 week of their collection.

In one embodiment of any of the methods described herein, the removed aliquots of the dissolution medium or release solution are stored at about 2-8° C. (until tested) and are tested by HPLC analysis to determine free and optionally total amikacin content within 1 week of aliquot collection.

During manufacturing, the in vitro methods described herein may be used to test individual batches of liposomal aminoglycoside formulations. For instance, one embodiment is a method for preparing a liposomal aminoglycoside formulation comprising aminoglycoside encapsulated liposomes. The method comprises:
(a) obtaining a manufactured batch of the liposomal aminoglycoside formulation comprising aminoglycoside encapsulated liposomes,
(b) determining the release profile of a sample of the formulation in the batch by any of the IVR methods described herein, and
(c) optionally, if the release profile meets a predetermined standard, incorporating some or all of the remaining batch of the liposomal aminoglycoside formulation into one or more dosage forms (e.g., vials).

### Liposomal Aminoglycoside Formulations

The liposomal aminoglycoside formulation includes an aminoglycoside, or a pharmaceutically acceptable salt thereof, encapsulated in a plurality of liposomes, and, optionally, aminoglycoside in free form (i.e., not encapsulated). In a preferred embodiment, the aminoglycoside is amikacin or a pharmaceutically salt thereof, such as amikacin sulfate.

The percent liposomal associated aminoglycoside refers to the percent of the aminoglycoside present in the liposomal aminoglycoside formulation which is encapsulated in the liposomes (based upon 100% total content of aminoglycoside, in any form, in the formulation). The total and free aminoglycoside content in a liposomal aminoglycoside formulation (such as pre- or post-nebulization) can be measured by high performance liquid chromatography (HPLC). HPLC in one embodiment, is carried out with evaporative light scattering detection (ELSD) or charged aerosol detection (CAD) to determine the total and free aminoglycoside content.

In one embodiment, the percent liposomal associated aminoglycoside of the formulation subjected to one of the IVR methods described herein ranges from about 70% to about 100%. In another embodiment, the percent liposomal associated aminoglycoside of the formulation subjected to one of the IVR methods described herein ranges from about 80% to about 100%. In yet another embodiment, the percent liposomal associated aminoglycoside in the liposomal aminoglycoside formulation (before performing the IVR method) ranges from about 80% to about 100%, about 80% to about 99%, about 90% to about 100%, about 90% to about 99%, or about 95% to about 99%. In one embodiment, at least about 95% of the aminoglycoside is liposomally associated prior to being subjected to one of the IVR methods described herein. In a further embodiment, at least about 97% of the aminoglycoside is liposomally associated prior to being subjected to one of the IVR methods described herein. The percent liposomal associated aminoglycoside of the formulation will vary when carrying out one of the IVR methods described herein depending on the starting material and the conditions employed for carrying out the particular IVR method.

In one embodiment, the liposomal aminoglycoside formulation has one of the following release profiles according to the IVR method described in Example 1. The release profile provided below can be an average of multiple samples from a single batch, or from a single sample from a batch. The sample, in one embodiment, can be pooled from multiple vials (such as 6 or 12 vials) originating from the same liposomal aminoglycoside batch.

| | Percentage (%) Aminoglycoside Released | | | | |
|---|---|---|---|---|---|
| | | | | Mean release | Median release |
| Time | | | | profile (multiple | profile (multiple |
| Point | | | | samples from | samples from |
| (hr) | Range 1 | Range 2 | Range 3 | single batch) | single batch) |
| 0.5 | from about 5 | from about 5 | from about 6 | | |
| | to about 15 | to about 12 | to about 11 | | |
| 1 | from about 16 | from about 16 | from about 18 | About 23 | About 23 |
| | to about 35 | to about 30 | to about 29 | | |
| 1.5 | from about 25 | from about 28 | from about 29 | | |
| | to about 50 | to about 48 | to about 46 | | |
| 2 | from about 35 | from about 40 | from about 42 | | |
| | to about 65 | to about 60 | to about 57 | | |
| 3 | from about 45 | from about 50 | from about 51 | About 59 | About 60 |
| | to about 75 | to about 70 | to about 68 | | |
| 4 | from about 50 | from about 55 | from about 57 | | |
| | to about 83 | to about 80 | to about 76 | | |
| 6 | from about 63 | from about 68 | from about 66 | | |
| | to about 85 | to about 85 | to about 83 | | |
| 12 | from about 68 | from about 70 | from about 73 | | |
| | to about 97 | to about 94 | to about 90 | | |
| 24 | from about 70 | from about 73 | from about 75 | About 84 | About 85 |
| | to about 100 | to about 100 | to about 100 | | |

% aminoglycoside released = [free aminoglycoside (mg/mL)/total aminoglycoside (mg/mL)] × 100%

In one embodiment, the liposomal aminoglycoside formulation (e.g., ALIS) releases, according to the IVR method described in Example 2, (i) no more than about 20% at the 0.5 hour time point, (ii) not more than about 35% at the 1.0 hour time point, (iii) not more than about 50% at the 1.5 hour time point, (iv) not more than about 65% at the 2.0 hour time point, (v) not more than about 75% at the 3.0 hour time point, (vi) not more than about 83% at the 4.0 hour time point, (vii) not more than about 84% at the 6.0 hour time point, (viii) not more than about 97% at the 12.0 hour time point, (ix) not less than about 70% at the 24 hour time point, or (x) any combination of any of the foregoing, where the percent aminoglycoside released=[free aminoglycoside (mg/mL)/total aminoglycoside (mg/mL)]×100%). The release profile provided can be an average of multiple samples from a single batch, or from a single sample from a batch. The sample, in one embodiment, can be pooled from multiple vials originating from the same liposomal aminoglycoside batch

In an alternative embodiment, the liposomal aminoglycoside formulation has one of the following release profiles according to the IVR method described in Example 2. The release profile provided below can be an average of multiple samples from a single batch, or from a single sample from a batch. The sample, in one embodiment, can be pooled from multiple vials originating from the same liposomal aminoglycoside batch.

| | Percentage (%) Aminoglycoside Released | | | | |
|---|---|---|---|---|---|
| | | | | Mean release | Mean release |
| Time | | | | profile (multiple | profile (multiple |
| Point | | | | samples from | samples from |
| (hr) | Range 1 | Range 2 | Range 3 | single batch) | single batch) |
| 0.5 | from about 2 | from about 2 | From about 4 | About 2 | About 5 |
| | to about 3 | to about 3 | to about 7 | | |
| 1 | from about 8 | from about 9 | From about 10 | About 10 | About 13 |
| | to about 12 | to about 12 | to about 14 | | |
| 1.5 | from about 28 | from about 28 | from about 29 | About 31 | About 31 |
| | to about 35 | to about 33 | to about 34 | | |
| 2 | from about 71 | from about 71 | from about 68 | About 74 | About 71 |
| | to about 80 | to about 77 | to about 76 | | |
| 2.5 | from about 86 | from about 90 | from about 88 | About 93 | About 92 |
| | to about 100 | to about 96 | to about 94 | | |
| 3 | from about 88 | from about 91 | from about 90 | About 94 | About 94 |
| | to about 100 | to about 100 | to about 96 | | |

% aminoglycoside released = [free aminoglycoside (mg/mL)/total aminoglycoside (mg/mL)] × 100%

In one embodiment, the liposomal aminoglycoside formulation (e.g., ALIS) releases, according to the IVR method described in Example 2, (i) not more than 20% aminoglycoside at the 0.5 hour time point, (ii) not more than 50% aminoglycoside at the 1.5 hour time point, (iii) not less than 80% aminoglycoside at the 3.0 hour time point, or (iv) any combination of any of the foregoing (where the percent aminoglycoside released=[free aminoglycoside (mg/mL)/total aminoglycoside (mg/mL)]×100%). The release profile provided can be an average of multiple samples from a single batch, or from a single sample from a batch. The sample, in one embodiment, can be pooled from multiple vials originating from the same liposomal aminoglycoside batch.

The liposomal aminoglycoside formulation may include one or more pharmaceutically acceptable excipients for inhalation formulations, such as solvents (preferably water), isotonicity agents (such as sodium chloride) and pH adjusters (such as sodium hydroxide).

The liposomal aminoglycoside formulation may be included in a vial.

One embodiment is a kit comprising a plurality of vials, each vial containing a liposomal aminoglycoside formulation comprising aminoglycoside encapsulated liposomes. The liposomal aminoglycoside formulation in each vial may be from a manufactured batch of liposomal aminoglycoside formulation, where the batch has been validated to have a predetermined in vitro release profile by an in vitro method described herein. In one embodiment, the kit comprises 28 of the vials. In another embodiment, the kit contains 7, 14, 21, 35, 42, 49, or 56 of the vials. The kit may further contain a nebulizer.

### Aminoglycosides

In one embodiment, the aminoglycoside in the liposomal aminoglycoside formulation is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin or verdamicin, a pharmaceutically acceptable salt of one of the foregoing, or any combination of any of the foregoing. In another embodiment, the aminoglycoside is selected from AC4437, dibekacin, K-4619, sisomicin, amikacin, dactimicin, isepamicin, rhodestreptomycin, arbekacin, etimicin, KA-5685, sorbistin, apramycin, framycetin, kanamycin, spectinomycin, astromicin, gentamicin, neomycin, sporaricin, bekanamycin, H107, netilmicin, streptomycin, boholmycin, hygromycin, paromomycin, tobramycin, brulamycin, hygromycin B, plazomicin, verdamicin, capreomycin, inosamycin, ribostamycin, vertilmicin, pharmaceutically acceptable salts thereof, and any combination of any of the foregoing. The aminoglycoside can be in free base or salt form. Where the aminoglycoside has one or more chiral centers, unless specified otherwise, each unique stereoisomer and mixtures of any combination of them (including racemic mixtures) are included. In cases in which the aminoglycosides have one or more unsaturated carbon-carbon double bonds, both the cis (Z) and trans (E) isomers are included herein. In cases where the aminoglycosides exist in tautomeric forms, such as keto-enol tautomers, each tautomeric form is contemplated as being included herein.

In one preferred embodiment, the aminoglycoside is amikacin or a pharmaceutically acceptable salt thereof, such as amikacin sulfate. Amikacin sulfate refers to the disulfate salt of amikacin, i.e., D-Streptamine, O-3-amino-3-deoxy-α-D-glucopyranosyl-(1→6)-O-[6-amino-6-deoxy-α-D-glucopyranosyl-(1→4)]-N¹-(4-amino-2-hydroxy-1-oxobutyl)-2-deoxy-, (S)-, sulfate (1:2) salt, with a chemical formula of C₂₂H₄₃N₅O₁₃·2H₂SO₄ and a molecular weight of 781.76.

The liposomes described herein may contain one or more aminoglycosides (such as two aminoglycosides). In one preferred embodiment, the liposomes comprise only one aminoglycoside, such as amikacin or a pharmaceutically acceptable thereof (e.g., amikacin sulfate).

### Liposomes

The liposomes described herein contain a lipid component comprising one or more lipids. The lipids can be synthetic, semi-synthetic or naturally-occurring, including phospholipids, tocopherols, sterols, fatty acids, negatively-charged lipids and cationic lipids. In one embodiment, the aminoglycoside is impermeable to the bilayer of the liposome.

In one embodiment of any of the methods described herein, the lipid component of the liposomes comprises, or consists of, one or more net neutral lipids.

In one embodiment, the lipid component of the liposomes comprises, or consists of, a net neutral phospholipid and cholesterol. In a further embodiment, the net neutral lipid is a phosphatidylcholine. The phosphatidylcholine, in one embodiment, is dipalmitoylphosphatidylcholine (DPPC).

In one embodiment, the lipid component of the plurality of liposomes comprises a phospholipid. The phospholipid, in one embodiment, is dipalmitoylphosphatidylcholine (DPPC), phosphatidylcholine (EPC), phosphatidylglycerol (PG), phosphatidylinositol (PI), phosphatidylserine (PS), phosphatidylethanolamine (PE), and phosphatidic acid (PA); the soya counterparts of the foregoing, such as soy phosphatidylcholine (SPC), SPG, SPI, SPS, SPE, and SPA; the hydrogenated egg and soya counterparts (e.g., HEPC and HSPC); phospholipids made up of ester linkages of fatty acids in the 2 and 3 of glycerol positions containing chains of 12 to 26 carbon atoms and different head groups in the 1 position of glycerol that include choline, glycerol, inositol, serine, or ethanolamine, as well as the corresponding phosphatidic acids. The carbon chains on these fatty acids can be saturated or unsaturated, and the phospholipid may be made up of fatty acids of different chain lengths and different degrees of unsaturation.

Other examples of lipids include, but are not limited to, dimyristoylphosphatidycholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleylphosphatidyl-ethanolamine (DOPE), mixed phospholipids such as palmitoylstearoylphosphatidyl-choline (PSPC), and single acylated phospholipids, for example, mono-oleoyl-phosphatidylethanolamine (MOPE).

In further embodiments, the lipid component comprises a sterol. In a further embodiment, the lipid component comprises a sterol and a phospholipid, or consists essentially of a sterol and a phospholipid, or consists of a sterol and a phospholipid. Sterols for use with the invention include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols can include tocopherols, esters of tocopherols including tocopherol hemi-succinates, salts of tocopherols including tocopherol hydrogen sulfates and tocopherol sulfates. The term “sterol compound” includes sterols and tocopherols.

In one embodiment, the lipid component includes dipalmitoylphosphatidylcholine (DPPC), a major constituent of naturally-occurring lung surfactant. In one embodiment, the lipid component comprises DPPC and cholesterol, or consists essentially of DPPC and cholesterol, or consists of DPPC and cholesterol. In a further embodiment, the DPPC and cholesterol have a mole ratio in the range of from about 19:1 to about 1:1, or about 9:1 to about 1:1, or about 4:1 to about 1:1, or about 2:1 to about 1:1, or about 1.86:1 to about 1:1. In even a further embodiment, the DPPC and cholesterol have a mole ratio of about 2:1 or about 1:1.

In a preferred embodiment, the lipid component comprises DPPC and cholesterol. For instance, the liposomes may contain amikacin sulfate and include DPPC and cholesterol as the lipid component. In a further embodiment, the aminoglycoside is amikacin, e.g., amikacin sulfate. In one embodiment, the aminoglycoside (e.g., amikacin) is impermeable to the bilayer of the liposome.

Suitable liposomal aminoglycoside formulations are described in U.S. Pat. Nos. 7,718,189, 8,226,975, 9,566,234, and 9,895,385, each of which is hereby incorporated by reference in its entirety for all purposes

In order to minimize dose volume and reduce patient dosing time, in one embodiment, liposomal entrapment of the aminoglycoside (e.g., the aminoglycoside amikacin) is highly efficient and in turn, the lipid to aminoglycoside weight ratio is as low a value as possible and/or practical while keeping the liposomes small enough to penetrate patient mucus and biofilms. In one embodiment, the lipid to aminoglycoside weight ratio (sometimes expressed as lipid: aminoglycoside) in the liposomal aminoglycoside formulation can be about 0.7 (lipid): 1.0 (aminoglycoside), from about 0.5 (lipid): 1.0 (aminoglycoside) to about 0.8 (lipid): 1.0 (aminoglycoside), from about 0.55 (lipid): 1.0 (aminoglycoside) to about 0.8 (lipid): 1.0 (aminoglycoside), from about 0.55 (lipid): 1.0 (aminoglycoside) to about 0.75 (lipid). 1.0 (aminoglycoside), or from about 0.6 (lipid): 1.0 (aminoglycoside) to about 0.8 (lipid): 1.0. In a further embodiment, the liposomes provided herein are small enough to effectively penetrate a bacterial biofilm. The sustained activity profile of the liposomal product can be regulated by the nature of the lipid membrane and by inclusion of other excipients in the composition.

The liposomal aminoglycoside formulation, in one embodiment, pre-nebulization, comprises liposomes with a mean diameter, that is measured by a light scattering method, of about 0.01 microns to about 3.0 microns, for example, in the range about 0.2 to about 1.0 microns. In one embodiment, the mean diameter of the liposomes in the composition is about 125 nm to about 360 nm, about 125 nm to about 350 nm, about 150 nm to about 350 nm, about 230 nm to about 280 nm, about 240 nm to about 280 nm, about 250 nm to about 280 nm or about 260 nm to about 280 nm. In a further embodiment, the mean diameter of the plurality of liposomes (pre-nebulization), as measured by light scattering is from about 200 nm to about 400 nm, or from about 250 nm to about 400 nm, or from about 250 nm to about 300 nm, or from about 200 nm to about 300 nm. In even a further embodiment, the mean diameter of the plurality of liposomes, as measured by light scattering is from about 260 to about 280 nm.

In one embodiment, the pH of the liposomal aminoglycoside formulation ranges from about 6.1 to about 7.1, such as from about 6.1 to about 6.8.

In one embodiment, the liposomes and liposomal aminoglycoside formulations described herein are manufactured by one of the methods set forth in U.S. Patent Application Publication Nos. 2013/0330400 and 2008/0089927 and U.S. Pat. No. 7,718,189, each of which is incorporated by reference in its entirety for all purposes. Certain methods for making liposomal aminoglycoside formulations are described in U.S. Patent Application Publication No. 2021/0015750 and PCT Publication No. WO 2019/213398, each of which is incorporated by reference in its entirety for all purposes.

In one embodiment, the liposomes can be formed by dissolving one or more lipids in an organic solvent (e.g., ethanol) forming a lipid solution, and mixing an aqueous solution of the aminoglycoside with the lipid solution to form the aminoglycoside coacervate. In one preferred embodiment, the lipid solution comprises a phospholipid and a sterol, e.g., DPPC and cholesterol.

Liposomes can be produced by sonication, extrusion, homogenization, swelling, electroformation, inverted emulsion or a reverse evaporation method. Bangham's procedure (J. Mol. Biol. 13:238-252, 1965) produces ordinary multilamellar vesicles (MLVs) Lenk et al. (U.S. Pat. Nos. 4,522,803, 5,030,453 and 5,169,637, each of which is incorporated by reference herein in their entireties), Fountain et al. (U.S. Pat. No. 4,588,578, incorporated by reference herein in its entirety) and Cullis et al. (U.S. Pat. No. 4,975,282, incorporated by reference herein in its entirety) disclose methods for producing multilamellar liposomes having substantially equal interlamellar solute distribution in each of their aqueous compartments. U.S. Pat. No. 4,235,871, incorporated by reference herein in its entirety, discloses formulation of oligolamellar liposomes by reverse phase evaporation. Each of the methods is amenable for preparing a liposomal aminoglycoside formulation for use with the present invention.

Unilamellar vesicles can be produced from MLVs by a number of techniques, for example, the extrusion techniques of U.S. Pat. Nos. 5,008,050 and 5,059,421, each of which is incorporated by reference herein in their entireties. Sonication and homogenization can be so used to produce smaller unilamellar liposomes from larger liposomes.

The liposome formulation of Bangham et al. (J. Mol. Biol. 13:238-252, 1965) involves suspending phospholipids in an organic solvent which is then evaporated to dryness leaving a phospholipid film on the reaction vessel. Next, an appropriate amount of aqueous phase is added, the mixture is allowed to “swell,” and the resulting liposomes which consist of multilamellar vesicles (ML Vs) are dispersed by mechanical means. This formulation provides the basis for the development of the small sonicated unilamellar vesicles described by Papahadjopoulos et al. (Biochim. Biophys. Acta. 135:624-638, 1967), and large unilamellar vesicles. The disclosure of each of the foregoing publications, including patents, are incorporated by reference herein in their entireties.

Techniques for producing large unilamellar vesicles (LUVs), such as, reverse phase evaporation, infusion procedures, and detergent dilution, can be used to produce liposomes for use in the liposomal aminoglycoside formulations provided herein. A review of these and other methods for producing liposomes may be found in the text Liposomes, Marc Ostro, ed., Marcel Dekker, Inc., New York, 1983, Chapter 1, which is incorporated herein by reference. See also Szoka, Jr. et al., (Ann. Rev. Biophys. Bioeng. 9:467, 1980), which is also incorporated herein by reference in its entirety for all purposes.

Other techniques for making liposomes include those that form reverse-phase evaporation vesicles (REV), see U.S. Pat. No. 4,235,871. Another class of liposomes that may be used is characterized as having substantially equal lamellar solute distribution. This class of liposomes is denominated as stable plurilamellar vesicles (SPLV) as defined in U.S. Pat. No. 4,522,803, and includes monophasic vesicles as described in U.S. Pat. No. 4,588,578, and frozen and thawed multilamellar vesicles (FATMLV) as described above. The disclosure of each of the foregoing patents are incorporated by reference herein in their entireties.

A variety of sterols and their water soluble derivatives such as cholesterol hemisuccinate have been used to form liposomes. See, e.g., U.S. Pat. No. 4,721,612. Mayhew et al., PCT Publication No. WO 85/00968, described a method for reducing the toxicity of drugs by encapsulating them in liposomes comprising alpha-tocopherol and certain derivatives thereof. Also, a variety of tocopherols and their water soluble derivatives have been used to form liposomes, see PCT Publication No. WO 87/02219 The disclosure of each of the foregoing patents and patent application publications are incorporated by reference herein in their entireties.

### Treatments

The liposomal aminoglycoside formulations prepared can be administered to treat various pulmonary infections, including mycobacterial infections (e.g., pulmonary infections caused by nontuberculous Mycobacterium, also referred to herein as nontuberculous mycobacterial (NTM) infections), such as those described in U.S. Pat. Nos. 7,544,369, 7,718,189, 8,226,975, 8,632,804, 8,642,075, 8,679,532, 8,802,137, 9,566,234, 9,827,317 and 9,895,385, each of which is hereby incorporated by reference in its entirety. In one embodiment, the liposomal aminoglycoside formulations are administered via inhalation. For example, the liposomal aminoglycoside formulations can be aerosolized, such as with a nebulizer, and administered via inhalation. Suitable methods for administering the liposomal aminoglycoside formulations with a nebulizer are described in U.S. Pat. No. 9,566,234, which is hereby incorporated by reference.

The liposomal aminoglycoside formulation may be administered via nebulization. For example, one or more of the methods disclosed in U.S. Pat. No. 9,566,234 or U.S. Pat. No. 9,895,385, the disclosure of each of which is incorporated by reference herein in its entirety, can be used to administer the liposomal aminoglycoside formulation. In one embodiment, the percent liposomal associated aminoglycoside post-nebulization is from about 50% to about 80%, from about 50% to about 75%, from about 50% to about 70%, from about 55% to about 75%, or from about 60% to about 70%. In another embodiment, the percent liposomal associated aminoglycoside post-nebulization is from about 65% to about 75%.

One embodiment is a method of treating a pulmonary infection in a patient comprising administering a therapeutically effective amount of a liposomal aminoglycoside formulation comprising aminoglycoside encapsulated liposomes to the patient, where the liposomal aminoglycoside formulation is from a manufactured batch of liposomal aminoglycoside formulations and validated to have a predetermined in vitro release profile, where the release profile is determined by an in vitro method described herein.

Among the pulmonary infections (such as in cystic fibrosis or bronchiectasis patients) that can be treated with the methods of the invention are Pseudomonas (e.g., P. aeruginosa, P. paucimobilis, P. putida, P. fluorescens, and P. acidovorans), staphylococcal, Methicillin-resistant Staphylococcus aureus (MRSA), streptococcal (including by Streptococcus pneumoniae), Escherichia coli, Klebsiella, Enterobacter, Serratia, Haemophilus, Yersinia pesos, Burkholderia pseudomallei, B. cepacia, B. gladioli, B. multivorans, B. vietnarniensis, and Mycobacterium tuberculosis infections. The liposomal aminoglycoside formulations can also be administered to treat, for instance, a pulmonary nontuberculous mycobacterial (NTM) infection such as pulmonary M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilusavense, M. gordonae, M. ulcerans, M. fortuitum or M. fortuitum complex (M. fortuitum and M. chelonae) infection.

In one embodiment, the patient is a cystic fibrosis (CF) patient having an NTM infection, such as that caused by MAC. In another embodiment, the patient is a non-CF patient having an NTM infection, such as that caused by MAC. MAC patients, in one embodiment, are refractory to prior treatment. Prior treatment, for example, comprises a combination of a macrolide antibiotic and ethambutol. The macrolide antibiotic in one embodiment, is azithromycin, clarithromycin, erythromycin, carbomycin A, josamycin, kitamycin, midecamycin, oleandomycin, solithromycin, spiramycin, troleandomycin, tylosin, roxithromycin, or a combination thereof. In another embodiment, prior treatment comprises a combination of a macrolide antibiotic, ethambutol and a rifamycin compound. The rifamycin compound, in a further embodiment, is rifampin or rifabutin. In another embodiment, prior treatment comprises a combination of a macrolide antibiotic selected from azithromycin, clarithromycin, erythromycin, carbomycin A, josamycin, kitamycin, midecamycin, oleandomycin, solithromycin, spiramycin, troleandomycin, tylosin, roxithromycin, or a combination thereof, ethambutol and a rifamycin compound.

In another embodiment, the patient treated with one of the methods presented herein is an NTM patient and is NTM-treatment naïve.

### EXAMPLES

The present invention will now be further described by way of the following non-limiting examples. In applying the disclosure of these examples, it should be kept clearly in mind that the examples are merely illustrative of the present invention and should not be construed as limiting the scope of the invention in any way as many variations and equivalents that are encompassed by the present invention will become apparent to those skilled in the art upon reading the present disclosure.

### Example 1

A method to determine the release of amikacin from ALIS over time is performed using USP dissolution Apparatus II (paddle method) and Float-A-Lyzer® dialysis devices (available from Repligen Corp. of Rancho Dominguez, CA, USA) with surfactant (5% Triton® X-100) in buffer (Dulbecco's Phosphate Buffered Saline (DPBS)) as a media. ALIS drug product is loaded in Float-A-Lyzer® dialysis devices and placed in the media. The constant temperature and mixing speed are achieved using a dissolution apparatus. Sample aliquots are removed from the vessels at specified timepoints for up to 24 hours, filtered via solid phase extraction to remove surfactant, and analyzed for amikacin content for the determination of the percentage amikacin release.

### Instrumentation and Equipment

Dialysis Device: 1000 kDa MWCO, 5-mL volume, Float-A-Lyzer G2 (cellulose ester dialysis membrane), Repligen, Cat. No. G235062.
Solid Phase Extraction (SPE) cartridge: Oasis MCX (1 cc/30 mg), Waters, Cat. No.

### Preparation of Solutions

All volumes may be adjusted as long as the ratios remain the same.
5.0% Triton X-100 in DPBS 1× (Media)

To prepare 6000 mL, slowly transfer 300-mL of Triton X-100 (BioXtra) into 5700 mL DPBS 1× buffer while mixing with a magnetic stir bar and stir plate.
10% Perfluoropentanoic Acid (PFPA) in H₂O

To prepare 10 mL, transfer 1-mL of PFPA into a 10-mL volumetric flask. Dilute to volume with HPLC Grade H₂O and mix well.
20% EtOH (H₂O: EtOH, 80:20, v/v)

To prepare 1000 mL, mix 200 mL of EtOH with 800 mL of deionized water.
Wash Solution (H₂O: MeOH: Formic Acid, 50:50:0.005, v/v)

To prepare 1000 mL, transfer 500 mL of HPLC Grade H₂O with 500 mL of methanol (MeOH) and 50 μL of formic acid into an appropriate container and mix well.
Elute Solution (0.05N NaOH in 20% Methanol)

To prepare 500 mL, pipette 2.5-mL of 10N NaOH into a suitable container containing 400 mL of HPLC Grade H₂O and 100 mL of MeOH. Mix well.
Diluent (H₂O: n-PrOH: PFPA, 70:30:0.3, v/v)

To prepare 1000 mL of diluent, transfer 700 mL deionized water, 300 mL n-propanol (n-PrOH) and 3 mL of PFPA to an appropriate container and mix well.
Amikacin liposome inhalation suspension (ALIS)

### Instrument Conditions

The USP dissolution Apparatus II is operated at 37° C. (±2° C.) and a paddle speed of 150 rpm (±10 rpm). The media is 5.0% v/v Triton X-100 (±0.2%) in DPBS 1× (900 mL). 5 mL samples are taken at the 1, 2, 6, and 24 hour time points.

### Assay Procedure

900 mL of media is transferred into six different vessels. Evaporation covers are placed on the vessels and equilibrated to 37° C. with 150 rpm paddle speed agitation.

Pre-treat Twelve Float-A-Lyzer dialysis devices were pretreated as follows: The caps of the dialysis devices were removed, and the devices were filled with 20% EtOH. The caps were replaced, and the dialysis devices were submerged in the same alcohol solution (20% EtOH) for 30 minutes. The devices were removed from the alcohol solution and the 20% EtOH solutions were emptied from the device. The interior and exterior of the devices were flushed thoroughly with a continuous stream of deionized water then the devices were filled with deionized water, caps replaced, and dialysis devices soaked in water for 30 minutes. The device was removed from the deionized water and the deionized water was emptied from within the device. The interior and exterior of the devices were then flushed with a continuous stream of deionized water. All remaining drops of deionized water were shaken and/or tapped out prior to loading the device.

ALIS vials (6 or 12 vials) were equilibrated to ambient temperature (minimum 45 minutes). Vials were shaken or Vortexed until the sample appeared uniform and well mixed. Each ALIS vial was assigned to a specific vessel and labeled accordingly.

The contents of each ALIS vial were poured directly into the dialysis devices (Float-A-Lyzers) by evenly distributing the contents into 2 paired pre-treated Float-A-Lyzers, each having a predetermined volume of 5 mL. Once the dialysis devices were filled, the caps on the devices were replaced. Each pair of Float-A-Lyzer was assigned to a single dissolution vessel. This process was repeated for all vials being tested.

Loaded Float-A-Lyzers were attached to the dissolution instrument's cover/adaptor assembly. The rotation of the paddles of the dissolution instrument was stopped, and each Float-A-Lyzer/Cover/Adaptor was placed into a vessel. It was ensured that the Float-A-Lyzers were not accidentally crimped when placing into vessels. The height of adaptor was adjusted if necessary, to ensure that the respective Float-A-Lyzer did not contact the paddle while remaining submerged in the dissolution media.

Dissolution is begun and samples (5 mL aliquots) are taken after 1, 2, 6, and 24 hours.

### Sample Solid Phase Extraction (SPE) Pre-Treatment

62.5 μL of 10% PFPA was pipetted into 5 mL sample pulls (aliquots) and gently vortexed until well mixed. SPE cartridges were loaded into the extraction manifold using long needle valves. Empty collection test tubes were placed into the extraction manifold test tube rack. Vacuum pressure of the extraction manifold was adjusted to a set point of 15 inHg.

One cartridge volume (˜1-mL) of HPLC Grade H₂O was transferred into each cartridge. All in-use long needle valves were opened, and the vacuum pump was turned on. The pump was shut off once all H₂O was passed through, and all long needle valves were closed.

1-mL of PFPA pre-treated sample was pipetted into each cartridge and was allowed to sit in the cartridge for 5 minutes. Open All in-use long needle valves were opened, and the vacuum pump was turned on. The pump was shut off once all sample was passed through.

Cartridges were washed with six cartridge volumes of wash solution (˜6 mL). Following wash, the pump was left on for twenty minutes to dry the cartridges. The pump was shut off upon completion, and all long needle valves were closed.

Waste test tubes were replaced with fresh, properly labeled collection test tubes. 0.5-mL of elute solution was pipetted into each cartridge and allowed to sit in each cartridge for 5 minutes. All in-use long needle valves were opened, and the vacuum pump was turned on. The pump was shut off after the elute solution had been passed through all cartridges. All long needle valves were closed, and elution procedure was repeated for a total of 1-mL elution. The test tubes were vortexed to mix the eluted solution.

### Sample Preparation for HPLC Analysis

Final dilutions from mixed eluted solution were made as per Table 1. It was ensured that the final sample dilutions were sufficiently mixed by vortexing.

TABLE 1
Final Sample Dilutions

| Time Point | Vol. of Eluted | Volume of | Final Volume | Dilution |
|---|---|---|---|---|
| (hour) | Solution (mL) | Diluent (mL) | (mL) | Factor |
| 1 | 0.350 | 0.650 | 1 | 2.893 |
| 2 | 0.200 | 0.800 | 1 | 5.0624 |
| 6 | 0.120 | 0.880 | 1 | 8.4375 |
| 24 | 0.100 | 0.900 | 1 | 10.125 |

Analyze all sample preparations.

Samples were re-diluted or concentrated as necessary to fit within calibration curve. Elute solution was vialed and injected as-is if necessary, to get as close to the calibration range as possible.

### Calculations

Calculate individual dissolution vessel percent release as follows:

$$1 - hr \% Release = \frac{[ C 1 hr ( \frac{mg}{mL} ) * 9 8.4 mL ]}{[ Label Claim ( mg ) ]} * 100$$

$$2 - hr \% Release = \frac{[ ( C 2 hr ( \frac{mg}{mL} ) * 9 3.4 mL ) + 5 mL * C 1 hr ( \frac{mg}{mL} ) ]}{[ Label Claim ( mg ) ]} * 100$$

$$6 - hr \% Release = \frac{[ ( C 6 hr ( \frac{mg}{mL} ) * 8 98.4 mL ) + 5 mL * ( C 1 hr ( \frac{mg}{mL} ) + C 2 hr ( \frac{mg}{mL} ) ]}{[ Label Claim ( mg ) ]} * 100$$

$$24 - hr \% Release = \frac{[ ( C 6 hr ( \frac{mg}{mL} ) * 8 98.4 mL ) + 5 mL * ( C 1 hr ( \frac{mg}{mL} ) + C 2 hr ( \frac{mg}{mL} ) + C 6 hr ( \frac{mg}{mL} ) ]}{[ Label Claim ( mg ) ]} * 100$$

where:

C1 hr=Vessel Concentration at 1-hour, C2 hr=Vessel Concentration at 2-hours, C6 hr=Vessel Concentration at 6-hours, and C24 hr=Vessel Concentration at 24-hours.

The percent release of six replicates was averaged for reportable results at each timepoint.

If the percent release of the individual replicates failed to conform with the requirements in Table 2, testing through L₂ to L₃ was continued as necessary.

TABLE 2
USP <711> Extended-Release Dosage Forms Requirements

| | Number | |
|---|---|---|
| Level | Tested | Criteria |
| L₁ | 6 | No individual value lies outside each of the stated ranges; no |
| | | individual value is less than stated amount at the final test time |
| L₂ | 6 | The average value of the 12 units (L₁ + L₂) lies within each of the |
| | | stated ranges and is NLT the stated amount at the final test time; |
| | | none is >10% of labeled content outside each of the stated ranges |
| | | and none is >10% of the labeled content below the stated amount |
| | | at the final test time. |
| L₃ | 12 | The average value of the 24 units (L₁ + L₂ + L₃) lies within each of |
| | | the stated ranges and is NLT the stated amount at the final test time; |
| | | NMT 2 of the 24 units are more than 10% of labeled content |
| | | outside each of the stated ranges; NMT 2 of the 24 units are >10% |
| | | of labeled content below the stated amount at the final test time; |
| | | and none of the units are >20% of labeled content outside each of |
| | | the stated ranges or >20% of the labeled content below the stated |
| | | amount at the final test time. |

### Example 2

### Materials

Triton™ X-100 is a non-ionic surfactant available from The Dow Chemical Company (Midland, MI). Triton X-100 refers to octylphenol ethoxylate (also known as polyethylene glycol tert-octylphenyl ether (CAS No. 9002-93-1)) having a cloud point of 66° C. (1 wt % active aqueous solution), an HLB of 13.4, a moles ethoxylate (EO) of 9.5, a pH in 5% aqueous solution of 6, a pour point of 1 (C), a viscosity at 25° C. of 240 cP, a density at 25° C. of 1.061 g/mL and a flash point (closed cup) according to ASTM D93 of 251° C.

Dulbecco's Phosphate-Buffered saline (DPBS, 1×, Corning®, without calcium and magnesium) is a water-based salt solution containing potassium chloride (0.2 g/L), potassium dihydrogen phosphate (0.2 g/L), sodium chloride (8 g/L) and disodium phosphate (1.15 g/L) available from ThermoFischer Scientific (Waltham, MA).

### Equipment

High performance liquid chromatography (HPLC): equipped with an evaporative light scattering detector (ELSD) (Sedex 85, Sedere).

HPLC Column; 3 μm particle size, 4.6 mm I.D.×150 mm length; Hypersil Gold, Thermo, Cat. No. 25003-154630

Filter: Centrisart I reverse centrifugal concentrators, 20,000 molecular weight cut off, Sartorius, Cat. No. 13249E

USP Dissolution Apparatus 2 (Paddle) with a small volume, round bottom dissolution vessel (150 mL)

### Determination of In-Vitro Release of Liposomal Amikacin for Inhalation (ALIS)

The assay for the percent release of amikacin over time for ALIS was performed using a step-wise dilution of ALIS in the presence of the octylphenol ethoxylate surfactant (Triton™ X-100, referred to as “Triton” in this Example for brevity) in buffer (Dulbecco's Phosphate Buffered Saline, pH 7.2). The percent leakage of amikacin from the liposome is achieved utilizing a USP Dissolution Apparatus 2 (Paddle) to maintain a constant temperature and agitation. Samples remain incubated and agitated for up to 3 hours; and sample aliquots are pulled every thirty minutes to characterize the release rate over time. Collected samples are analyzed for total and free amikacin content via high performance liquid chromatography with evaporative light scattering detection (ELSD) for the determination of percent amikacin release.

### 1) Formulation of Solutions

All volumes may be adjusted as long as the ratios remain the same.

### 200 ppm Triton X-100 in DPBS Buffer (Triton/PBS Buffer)

To prepare 500 mL, using a positive displacement pipette, 100 μL of Triton X-100 was transferred into 500 mL DPBS buffer, and mixed well.

### 1.5% Sodium Chloride (NaCl) in H₂O (1.5% NaCl):

To prepare 1 L of 1.5% NaCl, approximately 15.0 (±0.1) grams of NaCl was dissolved in 1000 mL of H₂O and mix well.

Diluent: 3:7 (v/v) n-Propyl Alcohol (n-PrOH)/H₂O with 0.3% Perfluoropentanoic Acid (PFPA):

To prepare 1 L of diluent, 700 mL H₂O, 300 mL n-PrOH and 3 mL PFPA were transferred to an appropriate container and mixed well.

Solvent A: 1:1 (v/v) n-PrOH: H₂O with 0.5% PFPA:

To prepare 1 L of solvent A, 500 mL of n-PrOH, 500 mL H₂O and 5 mL PFPA were transferred to an appropriate container and mixed well.

Mobile Phase: 65:35 (v/v) MeOH: H₂O with 0.3% PFPA

To prepare 1 L of mobile phase, 650 mL MeOH, 350 mL H₂O and 3 mL PFPA were transferred to an appropriate container and mixed well.

### 2) Instrument Conditions

### USP Dissolution Apparatus 2 (Paddle)

Speed: 150 (±20) RPM

Temperature: 37° C. (±3° C.)

### 3) Assay Procedure

### Sample Preparation

The Triton/PBS buffer was equilibrated in a dissolution vessel to 37° C. (agitation was not required, although can optionally be employed).

The samples were allowed to equilibrate to ambient temperature (minimum 45 min). The samples were shaken or vortexed until the samples appeared uniform and well mixed.

30 mL of ALIS was transferred into a separate dissolution vessel and allowed to equilibrate to 37° C. for approximately 1 hr with agitation.

Time=0 hour

0.5 mL of Triton/PBS buffer was added into 30 mL equilibrated ALIS sample to form an “ALIS mixture” in the dissolution vessel.

Time=0.5 hour

3 mL of ALIS mixture was removed from the vessel and transferred to a suitable labeled container. This sample is analyzed for total and free amikacin content. Each sample removed at t=0.5 to 3 hours can be stored at 2-8° C. for up to 1 week.

1.7 mL of Triton/PBS buffer was added into the remainder of ALIS mixture in the vessel, and incubated for 0.5 hour with agitation.

Time=1.0 hour

3 mL of ALIS mixture was removed from the vessel and transferred to a suitable labeled container. This sample is analyzed for total and free amikacin content.

4.0 mL of Triton/PBS buffer was added into the remainder of ALIS mixture in the vessel, and incubated for 0.5 hour with agitation.

Time=1.5 hour

3 mL of ALIS mixture was removed from vessel and transferred to a suitable labeled container. This sample is analyzed for total and free amikacin content.

16 mL of Triton/PBS buffer was added into the remainder of ALIS mixture in the vessel, and incubated for 0.5 hour with agitation.

Time=2.0 hour

20 mL of ALIS mixture was removed from vessel and transfer to a suitable labeled container. This sample will be analyzed for total and free amikacin content.

100 mL of Triton/PBS buffer was added into the remainder of ALIS mixture in the vessel, and incubated for 0.5 hour with agitation.

Time=2.5 hour

20 mL of ALIS mixture was removed from the vessel and transferred to a suitable labeled container. This sample is analyzed for total and free amikacin content.

The ALIS mixture was incubated and agitated for 0.5 hour.

Time=3.0 hour

20 mL of ALIS mixture was removed from the vessel and transferred to a suitable labeled container. This sample is analyzed for total and free amikacin content.

### 4) Sample Formulation for HPLC Analysis

### Total Amikacin Content

A positive displacement pipette was used to prepare a two-step dilution, first in Solvent A, then in Diluent for the collected samples from each time point as directed in Table 3. Samples expire in 2 weeks when stored at 2-8° C.

TABLE 3
Preparation of Samples for Total Amikacin Determination

| | Dilution A | | Dilution B | | |
|---|---|---|---|---|---|
| Time | ALIS | Total Volume | Dilution | Total volume | Final |
| Point | mixture | in Solvent A | A | in Diluent | Dilution |
| (hr) | (mL) | (mL) | (mL) | (mL) | Factor |
| 0.5 | 0.2 | 5 | 0.25 | 10 | 1000 |
| 1.0 | 0.2 | 5 | 0.25 | 10 | 1000 |
| 1.5 | 0.2 | 5 | 0.25 | 10 | 1000 |
| 2.0 | 0.2 | 5 | 0.25 | 5 | 500 |
| 2.5 | 0.2 | 5 | 0.25 | 1 | 100 |
| 3.0 | 0.2 | 5 | 0.25 | 1 | 100 |

### Free Amikacin Content

Dilution A: Dilutions of ALIS mixture in 1.5% NaCl were prepared as described in Table 4.

TABLE 4
Free Amikacin Determination - Dilution in 1.5% NaCl

| | | | Dilution |
|---|---|---|---|
| Time Point (hr) | ALIS mixture (mL) | Total in NaCl (mL) | Factor |
| 0.5 | 1 | 5 | 5 |
| 1.0 | 1 | 5 | 5 |
| 1.5 | 1 | 5 | 5 |
| 2.0 | 1 | 2 | 2 |
| 2.5 | 2.5 | — | 1 |
| 3.0 | 2.5 | — | 1 |

The Dilution A was transferred to a filter. For the Centrisart filter apparatus, the inner filter portion was removed and approximately 2.5 mL of Dilution A was transferred into the outer Centrisart tube. The inner tube was reinserted to contact the sample surface. The filter was allowed to remain in contact with the sample for at least 5 minutes.

The sample was centrifuged at 2500×g at ambient conditions for 15 minutes.

The filtrate was collected from the inner Centrisart tubes and stored in an appropriate container.

Dilutions B and C: Further dilutions of filtrate collected for each time point were prepared as shown in Tables 5 and 6. Samples (Dilution C) expire in 2 weeks when stored at 2-8° C.

TABLE 5
Free Amikacin Dilutions B and C - for Release Samples

| | Dilution B | | Dilution C | | |
|---|---|---|---|---|---|
| Time | | Total | Filtrate/ | Total | Final |
| Point | Filtrate | Volume in | Dil-B | Volume in | Dilution |
| (hr) | (mL) | Diluent (mL) | (mL) | Diluent (mL) | Factor |
| 0.5* | — | — | 0.125 | 5 | 200 |
| 1.0* | — | — | 0.1 | 5 | 250 |
| 1.5 | 0.2 | 2 | 0.2 | 2 | 500 |
| 2.0 | 0.2 | 2 | 0.1 | 2 | 400 |
| 2.5 | 0.2 | 2 | 0.2 | 2 | 100 |
| 3.0 | 0.2 | 2 | 0.2 | 2 | 100 |

TABLE 6
Free Amikacin Dilutions B and C -
for Stability Samples ≥3 Months.

| | Dilution B | | Dilution C | | |
|---|---|---|---|---|---|
| Time | | Total | Filtrate/ | Total | Final |
| Point | Filtrate | Volume in | Dil-B | Volume in | Dilution |
| (hr) | (mL) | Diluent (mL) | (mL) | Diluent (mL) | Factor |
| 0.5 | — | — | 0.2 | 2 | 50 |
| 1.0 | — | — | 0.2 | 5 | 125 |
| 1.5 | 0.2 | 2 | 0.2 | 2 | 500 |
| 2.0 | 0.2 | 2 | 0.1 | 2 | 400 |
| 2.5 | 0.2 | 2 | 0.2 | 2 | 100 |
| 3.0 | 0.2 | 2 | 0.2 | 2 | 100 |

All sample formulations were analyzed for total and free amikacin content via HPLC and ELSD, as follows.

### Standard Preparation

Amikacin Stock Standard Solution: 3.3 mg/mL amikacin in H₂O. Approximately 82.5 mg of Amikacin Reference Standard (USP, Cat. No. 1019508) was weighed and quantitatively transferred to a 25-mL volumetric flask. Amikacin was dissolved and Q.S. with H₂O, and mixed well. The actual amikacin concentration was calculated.

Amikacin Working Standard Solutions. Using a positive displacement pipette, a series of dilutions of the Amikacin Stock Standard Solution were prepared, in diluent, as outlined in Table 7, below.

TABLE 7
Amikacin working standard solutions

| Working | Volume stock | Total volume | Approximate |
|---|---|---|---|
| standard | (mL) | (mL) | concentration (μg/mL) |
| 1 | 0.100 | 10 | 33 |
| 2 | 0.135 | 10 | 45 |
| 3 | 0.170 | 10 | 56 |
| 4 | 0.200 | 10 | 66 |
| 5 | 0.240 | 10 | 79 |

Amikacin Check Stock Standard Solution. 3.3 mg/mL amikacin in H₂O. Prepared by the same procedure as the Amikacin Stock Standard Solution.

Check Standard Working Solution: 56 μg/mL amikacin in diluent. A dilution of the Amikacin Check Stock Standard Solution was prepared as per working standard 3 in Table 7.

Kanamycin Stock Solution: 3.0 mg/mL in H₂O. 30 mg Kanamycin Sulfate Reference Standard (USP, Cat. No. 1355006) was weighed and transferred to a 10-mL volumetric flask. The Reference Standard was dissolved in and diluted to volume with H₂O, and mixed well.

Resolution Solution: 56 μg/mL amikacin and 60 μg/mL kanamycin in diluent. 0.85 mL of Amikacin Stock or Check Stock Solution and 1.0 mL of Kanamycin Stock Solution were pipetted into a 50-mL volumetric flask, diluted to volume with diluent and mixed well.

### HPLC Settings

Isocratic flow rate: 1.1 mL/min (±0.1 mL/min)
Injection volume: 15 μL
Column Temperature: 30° C. (±3° C.)
Run Time: 7.5 minutes

### HPLC Injection Scheme

Diluent Blank: as many injections as necessary to obtain a clean baseline
Injection Precision (working standard 3): 6 injections
Resolution Solution: single injection
Linearity Standards (5 levels): 2 injections each from low to high
Check Working Standard: 2 injections (only when new stock standard is prepared)
Samples: 2 injections each (Up to six samples or twelve injections)
Linearity Standards (5 levels): 2 injections each from low to high

### ELSD Settings

These parameters are validated for the Sedex 85. The parameters may need to be altered if a different ELSD model is utilized.
Gain: 10
Drift Tube Temperature: 55° (±5°) C.
Pressure: 3.0 bar (±0.3 bar)
Filter: 5S

### 5) Calculations

% Amikacin Release is determined by:

$$\% Release = [ Free Amikacin ( mg / mL ) / Total Amikacin ( mg / mL ) ] \times 100 \%$$

A graph of % release vs. Time was plotted to represent the % release of amikacin over time.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

What is claimed is:

1. A method for evaluating the aminoglycoside release profile of a liposomal aminoglycoside formulation comprising an aminoglycoside encapsulated in a plurality of liposomes, the method comprising
(a) operating a dissolution apparatus in a dissolution vessel, the dissolution vessel containing (i) a predetermined quantity of the liposomal aminoglycoside formulation in one or more dialysis devices each having a predetermined volume and a dialysis membrane having a predetermined molecular weight cut off (MWCO), and (ii) a dissolution medium having a predetermined quantity of surfactant, wherein each dialysis membrane is permeable to the aminoglycoside, the dissolution medium, and the surfactant and substantially impermeable to the liposomes;
wherein the dissolution apparatus and the one or more dialysis devices are immersed in the dissolution medium;
(b) after a predetermined time interval, removing an aliquot of the dissolution medium;
(c) analyzing the aliquot for free aminoglycoside content; and
(d) optionally, repeating steps (b) and (c) after one or more additional time intervals.

2. The method of claim 1, wherein the method comprises, prior to operating the dissolution apparatus,
(a) adding a predetermined quantity of the liposomal aminoglycoside formulation into the one or more dialysis devices; and
(b) placing the one or more dialysis devices into the dissolution medium.

3. The method of claim 1 or 2, wherein the one or more dialysis devices each comprise a dialysis membrane having a molecular weight cut off (MWCO) of between about 20 kD and about 1500 kD.

4. The method of claim 3, wherein the one or more dialysis devices each comprise a dialysis membrane having a molecular weight cut off (MWCO) of about 1000 kD.

5. The method of any one of claims 1-4, wherein each dialysis membrane is comprised of cellulose ester.

6. The method of any one of claims 1-5, wherein the dissolution vessel contains two dialysis devices, each containing a portion of the liposomal aminoglycoside formulation.

7. The method of any one of claims 1-6, wherein the dissolution medium comprises a buffer.

8. The method of any one of claims 1-7, wherein the surfactant has a critical micelle concentration (CMC) less than 5 mM.

9. The method of any one of claims 1-8, wherein the surfactant has a CMC less than 1 mM.

10. The method of any one of claims 1-9, wherein the surfactant is a non-ionic surfactant.

11. The method of claim 10, wherein the non-ionic surfactant is octylphenol ethoxylate.

12. The method of claim 10, wherein the dissolution medium comprises octylphenol ethoxylate in phosphate-buffered saline.

13. The method of claim 10, wherein the dissolution medium comprises from about 0.5 to about 10% v/v of octylphenol ethoxylate in phosphate-buffered saline.

14. The method of claim 10, wherein the dissolution medium comprises from about 1 to about 6% v/v of octylphenol ethoxylate in phosphate-buffered saline.

15. The method of claim 10, wherein the dissolution medium comprises about 5% v/v of octylphenol ethoxylate in phosphate-buffered saline.

16. The method of any one of claims 12-15, wherein the phosphate-buffered saline is Dulbecco's phosphate-buffered saline.

17. The method of any one of claims 1-16, wherein operating the dissolution apparatus comprises agitating the dissolution medium with a paddle.

18. The method of claim 17, wherein the paddle is operated at from about 75 rpm to about 150 rpm.

19. The method of claim 17, wherein the paddle is operated at about 150 rpm.

20. The method of any one of claims 1-16, wherein operating the dissolution apparatus comprises agitating the dissolution medium with a stir bar.

21. The method of claim 20, wherein the stir bar is operated at from about 75 rpm to about 150 rpm.

22. The method of claim 20, wherein the stir bar is operated at about 150 rpm.

23. The method of any one of claims 1-22, wherein the dissolution apparatus is operated at 37° C.

24. The method of any one of claims 1-19, wherein the dissolution apparatus is a USP apparatus 2 (paddle) operated at about 150 rpm.

25. The method of any one of claims 1-24, wherein step (b) comprises performing solid phase extraction on the aliquot to remove surfactant prior to analyzing the aliquot for free aminoglycoside content.

26. The method of claim 16, wherein the solid phase extraction is performed with a cation exchange sorbent material.

27. The method of any one of claims 1-26, wherein aliquots of the dissolution medium are taken after 1, 2, 6, and 24 hours.

28. The method of any one of claims 1-26, wherein aliquots of the dissolution medium are taken after 1, 2, 3, 4, 6, and 24 hours.

29. The method of any one of claims 1-26, wherein aliquots of the dissolution medium are taken after 1, 2, 3, 6, and 24 hours.

30. The method of any one of claims 1-26, wherein aliquots of the dissolution medium are taken after 1, 2, 4, and 24 hours.

31. The method of any one of claims 1-26, wherein aliquots of the dissolution medium are taken after 1, 2, 4, 6 and 24 hours.

32. The method of any of claims 1-31, wherein the aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin or verdamicin, AC4437, K-4619, sisomicin, dactimicin, arbekacin, etimicin, KA-5685, sorbistin, apramycin, sporaricin, bekanamycin, H₁₀₇, boholmycin, paromomycin, tobramycin, brulamycin, hygromycin B, plazomicin, verdamicin, inosamycin, vertilmicin, a pharmaceutically acceptable salt thereof, or any combination of the forgoing.

33. The method of any one of claims 1-31, wherein the aminoglycoside is amikacin or a pharmaceutically acceptable salt thereof.

34. The method of any one of claims 1-31, wherein the aminoglycoside is amikacin sulfate.

35. The method of any one of claims 1-34, wherein the lipid component of the plurality of liposomes comprises a net neutral lipid.

36. The method of any one of claims 1-34, wherein the lipid component of the plurality of liposomes comprises a net neutral phospholipid and a sterol.

37. The method of claim 36, wherein the sterol is cholesterol.

38. The method of claim 36 or 37, wherein the net neutral lipid is a net neutral phosphatidylcholine.

39. The method of claim 38, wherein the net neutral phosphatidylcholine is dipalmitoylphosphatidylcholine (DPPC).

40. A method for measuring the release profile of aminoglycoside from a liposomal aminoglycoside formulation comprising an aminoglycoside encapsulated in a plurality of liposomes, the method comprising,
(a) adding a predetermined quantity of the liposomal aminoglycoside formulation in one dialysis device or divided into two or more dialysis devices, wherein each dialysis device has a predetermined volume and comprises a cellulose ester dialysis membrane;
(b) placing the dialysis devices into a dissolution vessel comprising a predetermined quantity of octylphenol ethoxylate in phosphate-buffered saline;
(c) operating a dissolution apparatus in the dissolution vessel, wherein the dissolution apparatus is a USP apparatus 2 (paddle) operated at 37° C. at about 150 rpm;
(d) after a predetermined time interval, removing an aliquot of the dissolution medium;
(e) analyzing the aliquot for free aminoglycoside content; and
(f) optionally, repeating steps (d) and (e) after one or more additional time intervals.

41. A method for preparing a liposomal aminoglycoside formulation comprising aminoglycoside encapsulated in a plurality of liposomes, the method comprising:
(a) obtaining a sample from a manufacturing batch of the liposomal aminoglycoside formulation comprising aminoglycoside encapsulated in a plurality of liposomes;
(b) evaluating the release profile of the sample comprising the steps of:
(i) adding a predetermined quantity of the liposomal aminoglycoside formulation in one dialysis device or divided into two or more dialysis devices, wherein each dialysis device has a predetermined volume and comprises a cellulose ester dialysis membrane;
(ii) placing the dialysis devices into a dissolution vessel comprising a predetermined quantity of octylphenol ethoxylate in phosphate-buffered saline;
(iii) operating a dissolution apparatus with the dissolution vessel, wherein the dissolution apparatus is a USP apparatus 2 (paddle) operated at 37° C. at about 150 rpm;
(iv) after a predetermined time interval, removing an aliquot of the dissolution medium;
(v) analyzing the aliquot for free aminoglycoside content; and
(vi) optionally, repeating steps (iv) and (v) after one or more additional time intervals; and
(c) optionally, if the release profile meets a predetermined standard, incorporating the batch of the liposomal aminoglycoside formulation into one or more dosage forms.

42. The method of claim 40 or 41, wherein the aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin or verdamicin, AC4437, K-4619, sisomicin, dactimicin, arbekacin, etimicin, KA-5685, sorbistin, apramycin, sporaricin, bekanamycin, H₁₀₇, boholmycin, paromomycin, tobramycin, brulamycin, hygromycin B, plazomicin, verdamicin, inosamycin, vertilmicin, a pharmaceutically acceptable salt thereof, a combination thereof, or any combination of the foregoing.

43. The method of claim 40 or 41, wherein the aminoglycoside is amikacin or a pharmaceutically acceptable salt thereof.

44. The method of claim 40 or 41, wherein the aminoglycoside is amikacin sulfate.

45. The method of any one of claims 40-44, wherein the lipid component of the plurality of liposomes comprises a net neutral lipid.

46. The method of any one of claims 40-44, wherein the lipid component of the plurality of liposomes comprises a net neutral phospholipid and a sterol.

47. The method of claim 46, wherein the sterol is cholesterol.

48. The method of claim 46 or 47, wherein the net neutral phospholipid is a net neutral phosphatidylcholine.

49. The method of claim 48, wherein the net neutral phosphatidylcholine is dipalmitoylphosphatidylcholine (DPPC).

50. The method of any one of claims 40-49, wherein the dialysis membrane has a molecular weight cut off (MWCO) of between about 20 kD and about 1500 kD.

51. The method of any one of claims 40-49, wherein the dialysis membrane has a molecular weight cut off (MWCO) of about 1000 kD.

52. The method of any one of claims 40-51, wherein the phosphate buffered saline is Dulbecco's phosphate buffered saline.

53. A method of treating a pulmonary infection in a patient comprising administering a therapeutically effective amount of a liposomal aminoglycoside formulation comprising aminoglycoside encapsulated liposomes to the patient, wherein the liposomal aminoglycoside formulation is from a manufactured batch of a liposomal aminoglycoside formulation and validated to have a predetermined in vitro release profile, where the release profile is determined by the method of any one of claims 1-52.

54. The method of claim 53, wherein the aminoglycoside is amikacin sulfate.

55. A kit comprising a plurality of vials, each vial containing a liposomal aminoglycoside formulation comprising aminoglycoside encapsulated in a plurality of liposomes, wherein the liposomal aminoglycoside formulation in each vial is from a manufactured batch, which has been validated to have a predetermined in vitro release profile, where the release profile is determined by the method of any one of claims 1-54.

56. A method for evaluating the release profile of a liposomal aminoglycoside formulation comprising aminoglycoside encapsulated in a plurality of liposomes, the method comprising,
(a) adding a predetermined quantity of surfactant to a predetermined quantity of the liposomal aminoglycoside formulation to obtain a release solution;
(b) after a predetermined time interval, removing an aliquot of the release solution and analyzing the aliquot for total and free aminoglycoside content;
(c) increasing the ratio of surfactant to the liposomal aminoglycoside formulation in the release solution;
(d) after a predetermined time interval, removing an aliquot of the release solution and analyzing the aliquot for total and free aminoglycoside content;
(e) optionally, repeating steps (c) and (d) one or more times; and
(f) optionally, repeating step (d) one or more times after one or more additional time intervals.

57. The method of claim 56, wherein the surfactant is added in the form of a dissolution medium comprising the surfactant and a buffer.

58. A method for evaluating the release profile of liposomal aminoglycoside formulation comprising aminoglycoside encapsulated liposomes, the method comprising:
(a) adding a predetermined quantity of a dissolution medium comprising a surfactant and a buffer to a predetermined quantity of the liposomal aminoglycoside formulation to obtain a release solution;
(b) after a predetermined time interval, removing an aliquot of the release solution and analyzing the aliquot for total and free aminoglycoside content;
(c) adding a predetermined quantity of the dissolution medium to the release solution;
(d) after a predetermined time interval, removing an aliquot of the release solution and analyzing the aliquot for total and free aminoglycoside content;
(e) optionally repeating steps (c) and (d) one or more times; and
(f) optionally, repeating step (d) one or more times after one or more additional time intervals.

59. The method of any of one of claims 56-58, wherein the predetermined quantity of liposomal aminoglycoside formulation is a predetermined quantity that is pooled from two or more vials of the formulation originating from a single manufacturing batch of the formulation.

60. The method of any one of claims 56-59, wherein the surfactant is a non-ionic surfactant.

61. The method of any one of claims 56-59, wherein the surfactant is an anionic surfactant.

62. The method of any one of claims 56-61, wherein the surfactant has a critical micelle concentration (CMC) less than 5 mM.

63. The method of any one of claims 56-62, wherein the surfactant has a CMC less than 1 mM.

64. The method of claim 60, wherein the non-ionic surfactant is octylphenol ethoxylate.

65. A method for measuring the release profile of amikacin from a liposomal amikacin formulation comprising amikacin encapsulated in a plurality of liposomes comprising, in a USP apparatus 2 (paddle) at 37° C. with a stirrer at about 150 rpm,
(a) adding about 0.5 mL of a dissolution medium into about 30 mL of the liposomal amikacin formulation in a dissolution apparatus to form a release solution;
(b) 30 minutes after step (a) [time=0.5 hour], (i) removing and analyzing about a 3 mL aliquot of the release solution for total and free amikacin content, and (ii) adding about 1.7 mL of dissolution medium to the remainder of the release solution;
(c) 30 minutes after step (b) [time=1 hour], (i) removing and analyzing about a 3 mL aliquot of the release solution for total and free amikacin content, and (ii) adding about 4.0 mL of the dissolution medium to the remainder of the release solution;
(d) 30 minutes after step (c) [time=1.5 hours], (i) removing and analyzing about a 3 mL aliquot of the release solution for total and free amikacin content, and (ii) adding about 16 mL of the dissolution medium to the remainder of the release solution;
(e) 30 minutes after step (d) [time=2 hours], (i) removing and analyzing about a 20 mL aliquot of the release solution for total and free amikacin content, and (ii) adding about 100 mL of the dissolution medium to the remainder of the release solution;
(f) 30 minutes after step (e) [time=2.5 hours], removing and analyzing about a 20 mL aliquot of the release solution for total and free amikacin content; and
(g) 30 minutes after step (f) [time=3 hours], removing and analyzing about a 20 mL aliquot of the release solution for total and free amikacin content,
wherein the dissolution medium comprises octylphenol ethoxylate in phosphate-buffered saline.

66. The method of claim 65, wherein the phosphate-buffered saline is Dulbecco's phosphate-buffered saline.

67. The method of claim 65 or 66, wherein the about 30 mL of liposomal amikacin formulation in step (a) is pooled from two or more vials of the formulation originating from a single manufacturing batch of the formulation.

68. The method of any one of claims 56-67, wherein the dissolution medium comprises 200 ppm of octylphenol ethoxylate having an ethoxylate moles content of 9.5 in phosphate-buffered saline.

69. The method of claim 68, wherein the phosphate-buffered saline is Dulbecco's phosphate-buffered saline.

70. The method of any one of claims 56-69, wherein the aliquots are stored at 2-8° C. prior to analyzing, and analyzing for total and free aminoglycoside content comprises high-performance liquid chromatography (HPLC) analysis within 1 week of its storage.

71. The method of any one of claims 56-70, wherein prior to step (a), the formulation is equilibrated to 37° C.

72. The method of any one of claims 56-71, wherein the lipid component of the plurality of liposomes comprises a net neutral lipid.

73. The method of any one of claims 56-71, wherein the lipid component of the plurality of liposomes comprises a net neutral phospholipid and a sterol.

74. The method of claim 73, wherein the sterol is cholesterol.

75. The method of claim 73 or 74, wherein the net neutral phospholipid is a net neutral phosphatidylcholine.

76. The method of claim 75, wherein the net neutral phosphatidylcholine is dipalmitoylphosphatidylcholine (DPPC).

77. The method of any one of claims 56-64 and 68-76, wherein the aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin or verdamicin, AC4437, K-4619, sisomicin, dactimicin, arbekacin, etimicin, KA-5685, sorbistin, apramycin, sporaricin, bekanamycin, H₁₀₇, boholmycin, paromomycin, tobramycin, brulamycin, hygromycin B, plazomicin, verdamicin, inosamycin, vertilmicin, a pharmaceutically acceptable salt thereof, or any combination of the foregoing.

78. The method of any one of claims 56-64 and 68-76, wherein the aminoglycoside formulation comprises amikacin or a pharmaceutically acceptable salt thereof.

79. The method of claim 78, wherein the aminoglycoside formulation comprises amikacin sulfate.

80. A method for preparing a liposomal aminoglycoside formulation comprising aminoglycoside encapsulated liposomes, the method comprising:
(a) obtaining a sample from a manufacturing batch of the liposomal aminoglycoside formulation comprising aminoglycoside encapsulated in a plurality of liposomes;
(b) evaluating the release profile of the sample comprising the steps of:
(i) adding a predetermined quantity of dissolution medium comprising a surfactant and a buffer to the sample to obtain a release solution;
(ii) after a predetermined time interval, removing a portion of the release solution and analyzing the portion for total and free aminoglycoside content;
(iii) increasing the ratio of surfactant to the encapsulated aminoglycoside in the release solution;
(iv) after a predetermined time interval, removing a portion of the release solution and analyzing the portion for total and free aminoglycoside content;
(v) optionally, repeating steps (iii) and (iv) one or more times; and
(vi) optionally, repeating step (iv) one or more times; and
(c) optionally, if the release profile meets a predetermined standard, incorporating the batch of the liposomal aminoglycoside formulation into one or more dosage forms.

81. The method of claim 80, wherein the aminoglycoside formulation comprises amikacin or a pharmaceutically acceptable salt thereof.

82. The method of claim 80, wherein the aminoglycoside formulation comprises amikacin sulfate.

83. The method of any one of claims 80-82, wherein the sample in step (a) is obtained by pooling from two or more vials of the formulation originating from a single manufacturing batch of the formulation.

84. A method of treating a pulmonary infection in a patient comprising administering a therapeutically effective amount of a liposomal aminoglycoside formulation comprising aminoglycoside encapsulated in a plurality of liposomes to the patient, wherein the liposomal aminoglycoside formulation is from a manufactured batch of liposomal aminoglycoside formulations and validated to have a predetermined in vitro release profile, where the release profile is determined by:
(i) adding a predetermined quantity of dissolution medium comprising a surfactant and a buffer to a sample of liposomal aminoglycoside formulation from the batch to obtain a release solution;
(ii) after a predetermined time interval, removing a portion of the release solution and analyzing the portion for total and free aminoglycoside content;
(iii) increasing the ratio of surfactant to the encapsulated aminoglycoside in the release solution;
(iv) after a predetermined time interval, removing a portion of the release solution and analyzing the portion for total and free aminoglycoside content;
(v) optionally, repeating steps (iii) and (iv) one or more times; and
(vi) optionally, repeating step (iv) one or more times; and
(c) administering a therapeutically effective amount of liposomal aminoglycoside from the batch to the patient.

85. The method of claim 84, wherein the aminoglycoside formulation comprises amikacin sulfate.

86. The method of claim 84 or 85, wherein the sample in step (i) is obtained by pooling from two or more vials of the formulation originating from a single manufacturing batch of the formulation.

87. A kit comprising a plurality of vials, each vial containing a liposomal aminoglycoside formulation comprising aminoglycoside encapsulated in a plurality of liposomes, wherein the liposomal aminoglycoside formulation in each vial is from a manufactured batch, which has been validated to have a predetermined in vitro release profile, where the release profile is determined by:
(i) adding a predetermined quantity of dissolution medium comprising a surfactant and a buffer to a sample of liposomal aminoglycoside formulation from the batch to obtain a release solution;
(ii) after a predetermined time interval, removing a portion of the release solution and analyzing the portion for total and free aminoglycoside content;
(iii) increasing the ratio of surfactant to the encapsulated aminoglycoside in the release solution;
(iv) after a predetermined time interval, removing a portion of the release solution and analyzing the portion for total and free aminoglycoside content;
(v) optionally, repeating steps (iii) and (iv) one or more times; and
(vi) optionally, repeating step (iv) one or more times.

88. The kit of claim 87, wherein the aminoglycoside formulation comprises amikacin sulfate.

89. The kit of claim 87 or 88, wherein the kit comprises 28 of the vials.

90. The kit of any one of claims 87-89, wherein the sample in step (i) is obtained by pooling from two or more vials of the formulation originating from a single manufacturing batch of the formulation.

91. A liposomal amikacin formulation characterized by the in vitro release method of claim 65, wherein,
(i) the free amikacin content as measured in step (b) is no more than 20%,
(ii) the free amikacin content as measured in step (d) is no more than 50%,
(iii) the free amikacin content as measured in step (g) is no less than 80%, or
(iv) any combination of any of the foregoing.

92. A liposomal amikacin formulation characterized by the in vitro release method of claim 65, wherein,
(i) the free amikacin content as measured in step (b) ranges from about 2 to about 3%,
(ii) the free amikacin content as measured in step (c) ranges from about 8 to about 12%,
(iii) the free amikacin content as measured in step (d) ranges from about 28 to about 35%,
(iv) the free amikacin content as measured in step (e) ranges from about 71 to about 80%,
(v) the free amikacin content as measured in step (f) ranges from about 86 to about 100%, and
(vi) the free amikacin content as measured in step (g) ranges from about 88 to about 100%.

93. A liposomal amikacin formulation characterized by the in vitro release method of claim 65, wherein,
(i) the free amikacin content as measured in step (b) ranges from about 4 to about 7%,
(ii) the free amikacin content as measured in step (c) ranges from about 10 to about 14%,
(iii) the free amikacin content as measured in step (d) ranges from about 29 to about 34%,
(iv) the free amikacin content as measured in step (e) ranges from about 68 to about 76%,
(v) the free amikacin content as measured in step (f) ranges from about 88 to about 94%, and
(vi) the free amikacin content as measured in step (g) ranges from about 90 to about 96%.
